(19) United States
(12) Patent Application Publication
(10) Pub. No.: US 2023/0398098 A1
(43) Pub. Date: Dec. 14, 2023

(54) TOPICAL DETOMIDINE FORMULATIONS

(71) Applicant: Clexio Biosciences Ltd., Jerusalem (IL)

(72) Inventors: Mark ZAMANSKY, Jerusalem (IL); Anton FRENKEL, Jerusalem (IL)

(73) Assignee: Clexio Biosciences Ltd., Jerusalem (IL)

(21) Appl. No.: 18/455,242

(22) Filed: Aug. 24, 2023

Related U.S. Application Data
(63) Continuation of application No. 17/153,611, filed on Jan. 20, 2021.
(60) Provisional application No. 62/964,191, filed on Jan. 22, 2020.

(51) Int. Cl. A61K 31/4164; A61K 9/08; A61K 47/10; A61K 9/00

(52) U.S. Cl. CPC A61K 31/4164; A61K 9/08; A61K 47/10; A61K 9/0014

(57) ABSTRACT

The present disclosure relates to a purified detomidine HCl pharmaceutical product and to methods of preparation, validation of pharmaceutically acceptable product and use thereof.

### CROSS REFERENCE TO RELATED APPLICATIONS

This application is a continuation of U.S. Ser. No. 17/153,611, filed Jan. 20, 2021, which claims the benefit of U.S. Provisional Application No. 62/964,191, filed Jan. 22, 2020, the entire contents of both of which are incorporated by herein by reference.

### TECHNICAL FIELD

The present disclosure relates to formulations that contain detomidine and methods of treating pain using such formulations.

### BACKGROUND

### Detomidine

Detomidine, 4-[(2,3-dimethylphenyl)methyl]-1H-Imidazole, is an α-2-androgenic agonist available under the brand name Equimidine® and Dormosedan® for use as a veterinary sedative. Detomidine is not currently approved for human use.

Detomidine and related compounds, including its 3,4 dimethyl isomer, iso-detomidine (4-(3,4-Dimethylbenzyl)-1H-imidazole) were first described in U.S. Pat. No. 4,443,466. Both the '466 patent and the later U.S. Pat. No. 4,584,383 describe the synthetic method of manufacturing detomidine as being based on coupling of an imidazole moiety with 1-Bromo-2,3-dimethyl benzene using a Grignard reaction. RU2448095 describes an alternative route of synthesis of the molecule based on coupling in presence of a Titanium catalyst. According to both the '383 and '095 patents, detomidine is purified by crystallization of its hydrochloride salt from water. The chemical structures of detomidine HCl and iso-detomidine are shown below:

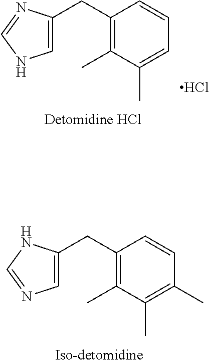

Two solid state forms of detomidine HCl are known, the anhydrous and monohydrate forms. Synthesis of the anhydrous form by crystallization of the monohydrate and further decomposition at elevated temperatures is described in U.S. Pat. No. 7,728,147. Synthesis of the anhydrous form via decomposition of the monohydrate in reduced pressure is described in Lane et al (1983). According to Veldre et al (2011), the anhydrous and monohydrate forms of detomidine HCl can easily interconvert depending on temperature and humidity.

The European Pharmacopeia 9.0 monograph (January 2014) describes detomidine HCl for veterinary use. The monograph lists the established HPLC method for identification of detomidine and its impurities as using a Symmetry C8, 5 μm, 4.6×150 mm column, with a mobile phase of Ammonium phosphate buffer pH 7.9-65% and Acetonitrile-35% at a flow rate of 1.0 mL/min and UV detection at 220 nm. That procedure is listed as recording three distinct impurities of detomidine:

### Impurity A: (RS)-(2,3-dimethylphenyl)(1H-imidazol-4-yl)methanol

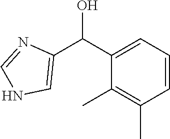

### Impurity B: (RS)-(1-benzyl-1H-imidazol-5-yl)(2,3-dimethylphenyl)methanol

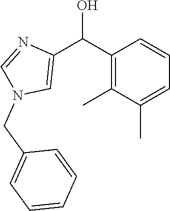 [SMILES: Cc1cccc(C(O)c2cn(Cc3ccccc3)cn2)c1C]

### Impurity C: 4-[(2,3-dimethylcyclohexyl)methyl]-1H-imidazole

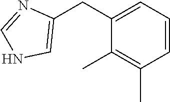

PCT/US18/012579 discloses topical formulations of detomidine and their uses in treating pain.

Purified detomidine for use in human pharmaceutical formulations is not known in the art.

### SUMMARY OF THE DISCLOSURE

It has now been identified that commercially available detomidine HCl products contain a variety of previously unidentified impurities. Provided herein are topical formulations of detomidine HCl substantially free of such impurities.

In some embodiments the formulations comprise detomidine or a pharmaceutically acceptable salt thereof, e.g. detomidine HCl, substantially free of iso-detomidine and/or iso-impurity A ((RS)-(3,4-dimethylphenyl)(1H-imidazol-4-yl)methanol).

In some embodiments, the formulations comprise detomidine or a pharmaceutically acceptable salt thereof, e.g. detomidine HCl, substantially free of 3-((1H-Imidazol-4-yl)methyl)-2-methylbenzaldehyde (“aldehyde impurity”).

In some embodiments, the formulations comprise detomidine HCl. In some embodiments the detomidine HCl is the monohydrate form. In some embodiments the detomidine HCl is the anhydrate form.

In some embodiments, the total amount of impurities identified within the formulations is not more than 0.1% area, not more than 0.06% area, or not more than 0.02% area, relative to detomidine, based on HPLC, using UV detection at 220 nm. In some embodiments, the total amount of impurities is not more than 0.06% area, relative to detomidine, based on HPLC, using UV detection at 220 nm.

In some embodiments, formulations comprise detomidine or a pharmaceutically acceptable salt thereof, e.g. detomidine HCl, substantially free of iso-impurity A (((RS)-(3,4-dimethylphenyl)(1H-imidazol-4-yl)methanol)). In some embodiments, the detomidine or a pharmaceutically acceptable salt thereof, e.g. detomidine HCl, is further substantially free of impurity A ((RS)-(2,3-dimethylphenyl)(1H-imidazol-4-yl)methanol). In some embodiments, the detomidine or a pharmaceutically acceptable salt thereof, e.g. detomidine HCl, is further substantially free of impurity B ((RS)-(1-benzyl-1H-imidazol-5-yl)(2,3-dimethylphenyl)methanol) and/or impurity C (4-[(2,3-dimethylcyclohexyl)methyl]-1H-imidazole).

Further provided are methods of treating a human subject in need thereof, comprising administering to the subject a formulation of the present invention. In some embodiments the human subject is in need of an analgesic. In some embodiments, the human subject is in need of an anti-pruritic agent.

Further provided are topical formulations comprising about 0.001 to about 3 wt % detomidine or a salt thereof and a carrier that is suitable for topical administration to a human subject's skin, wherein the carrier optionally comprises a water-miscible solubilizer that is present in an amount of 0.1 to 40% by weight of the formulation, wherein the formulation has a pH of 4.5 to 9 and wherein said topical formulation comprises less than 4% of the total detomidine content as crystalline detomidine base.

### BRIEF DESCRIPTION OF THE DRAWINGS

FIG. 1: DVS isotherm plot showing hydration of detomidine free base at 24.4° C.

FIG. 2: DVS isotherm plot showing hydration of detomidine HCl at 24.7° C.

FIG. 3: HPLC chromatogram showing impurity in sourced samples of detomidine HCl.

FIG. 4: HPLC chromatogram showing identification of the impurity peaks.

FIG. 5: LC-MS/MS identifying one of the impurity peaks as iso-impurity A.

FIG. 6: HR-ESI-MS identifying one of the impurity peaks as 3-((1H-Imidazol-4-yl)methyl)-2-methylbenzaldehyde.

FIG. 7: XRPD pattern of detomidine HCl monohydrate (sample 1).

FIG. 8: DSC thermogram of detomidine HCl monohydrate (sample 1).

FIG. 9: TGA thermogram of detomidine HCl monohydrate (sample 1).

FIG. 10: XRPD pattern of detomidine HCl monohydrate (sample 2).

FIG. 11: DSC thermogram of detomidine HCl monohydrate (sample 2).

FIG. 12: TGA thermogram of anhydrous Detomidine HCl (sample 2).

FIG. 13: microphotograph of particle morphology of detomidine HCl monohydrate prepared according to the second procedure (Carbon treatment and detomidine free base isolation, followed by crystallization).

FIG. 14: microphotograph of particle morphology of detomidine HCl monohydrate (sample 3).

FIG. 15: XRPD pattern of detomidine free base (sample “70”).

FIG. 16: DSC thermogram of detomidine free base.

FIG. 17: TGA thermogram of detomidine free base.

FIG. 18: photograph of formulation B showing foreign particles.

FIG. 19: photograph of isolated foreign particle from formulation B.

FIG. 20: photograph of opened sample tubes containing scaled up formulations A and B alongside placebo.

### DETAILED DESCRIPTION OF THE DISCLOSURE

The present invention is based, in part, on the identification of impurities present in detomidine preparations. The present invention is also based on methods of synthesizing and crystalizing detomidine to reduce the presence of the impurities. The present invention further provides methods to prepare purified solid detomidine HCl for use as a drug substance.

Anhydrous detomidine HCl is available under the brand name, inter alia, Equimidine® and Dormosedan® as a veterinary sedative. To date, detomidine has not been approved for human use.

In the present disclosure the singular forms “a”, “an” and “the” include the plural reference, and reference to a particular numerical value includes at least that particular value, unless the context clearly indicates otherwise. For example, “the method” includes the broadest definition of the meaning of the phrase, which can be more than one method.

By any range disclosed herein, it is meant that all hundredth, tenth and integer unit amounts within the range are specifically disclosed as part of the invention. Thus, for example, 0.01 mg to 50 mg means that 0.02, 0.03 . . . 0.09; 0.1, 0.2 . . . 0.9; and 1, 2 . . . 49, 50 mg unit amounts are included as embodiments of this invention.

A characteristic of a compound refers to a quality that the compound exhibits, as determined by for example, nuclear magnetic resonance (NMR) spectroscopy (nMS), mass spectroscopy (MS), infrared (IR), ultraviolet (UV) or fluorescence spectrophotometry, gas chromatography (GC), thin layer chromatography (TLC), high performance liquid chromatography (HPLC), elemental analysis, microscopic analysis, Ames test, and includes without limitation peaks, dissolution, stability, crystal shape, particle size, and any other quality that can be determined by an analytical method. Once the characteristics of a compound are known, the information can be used to, for example, screen or test for the presence of the compound in a sample and validate or reject a batch, for example a pharmaceutical batch.

As used herein, “substantially free” refers to a compound or composition having 0.01% area or less of a particular impurity or degradant, or having 0.1% area or less, 0.09% area or less, 0.08% area or less, 0.07% area or less, 0.06% area or less, 0.05% area or less, 0.04% area or less, 0.03% area or less, 0.02% area or less or 0.01% area or less, of total impurities or degradants, relative to detomidine, each as determined by HPLC, using UV detection at 220 nm. In preferred embodiments, the identification of impurities in the human detomidine drug substance is identified by the exemplary HPLC method, as disclosed herein.

As used herein, a “pharmaceutically acceptable” carrier or excipient is one that is suitable for use with humans and/or animals without undue adverse side effects (such as toxicity, irritation, and allergic response) commensurate with a reasonable benefit/risk ratio.

As used herein, “drug substance” refers to the active ingredient in a drug product, which provides pharmacological activity, prior to its incorporation into a drug product, in the treatment or prevention of a symptom or a disease, in a mammal, preferably a human.

As used herein, “drug product” refers to the dosage form containing the drug substance as well as at least one pharmaceutically acceptable carrier or excipient.

As used herein, a “composition” is distinct from a “pharmaceutical composition”, in that it does not include a pharmaceutically acceptable carrier or excipient. A composition as used herein is understood to be present in an inert environment. As used herein, a composition that is “free” of a chemical means that the composition contains, if at all, an amount of the chemical entity which cannot be avoided following an affirmative action intended to eliminate the presence of the chemical in the composition.

As used herein, “about” in the context of a numerical value or range means within ±10% of the numerical value or range recited or claimed.

As used herein, to “treat”, “treatment” or “treating” encompasses, e.g., reducing a symptom, inducing inhibition, regression, or stasis of the disorder and/or disease.

“Administering to the human subject” means the giving of, dispensing of, or application of medicines, drugs, or remedies to a subject/patient to relieve, cure, or reduce the symptoms associated with a condition, e.g., a pathological condition. The administration can be periodic administration.

As used herein, “suitable for topical administration” refers to compositions or formulations that are generally safe for administration to human skin.

“Detomidine” refers to the compound, 4-[(2,3-dimethylphenyl)methyl]-1H-imidazole, and to salts and hydrates, thereof. Detomidine is identified by CAS 76631-46-4 and detomidine HCl by CAS 90038-01-0.

Detomidine can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit is preferably in a form suitable for topical administration. Detomidine can be administered alone but is generally mixed with a pharmaceutically acceptable carrier.

The present disclosure relates, inter alia, to topical formulations comprising about 0.001 to about 3 wt % detomidine or a salt thereof, and methods of topical administration of such formulations wherein said detomidine or a salt thereof, is substantially free of 3-((1H-Imidazol-4-yl)methyl)-2-methylbenzaldehyde.

The present disclosure relates, inter alia, to topical formulations comprising about 0.001 to about 3 wt % detomidine or a salt thereof and, a carrier that is suitable for topical administration to a human subject's skin, wherein the carrier optionally comprises a water-miscible solubilizer that is present in an amount of 0.1 to 40% by weight of the formulation, wherein the formulation has a pH of 5.0 to 8.5 and wherein said topical formulation does not comprise crystalline detomidine base.

Pain is perhaps the most common symptom accompanying nearly every medical condition that a human can experience, and certain forms of pain, including those deriving from peripheral neuropathy, remain difficult to treat. Pruritus is an unpleasant sensation that provokes the desire to scratch. The condition is extremely common with estimates that at any given time between 8 and 16% of adults are suffering from it, resulting in significant reductions in quality of life for sufferers. The present inventors have developed topical formulations containing detomidine or a salt thereof that provide relief from pain and/or pruritus over an extended period of time. While not intending to be bound to any particular theory of operation, in certain instances, this beneficial effect may result at least partially from the fact that the formulations induce the formation of a depot of the detomidine or salt thereof in the subject's skin, thereby permitting a prolonged effect by release of the drug from the depot over time, and minimizing or avoiding systemic influence of the drug. These and other advantages that are conferred by the present formulations and methods using such formulations are described more fully herein.

As described above, the present disclosure pertains to topical formulations comprising detomidine or a salt thereof that are effective for treating pain and/or pruritus in a human subject. While not intending to be bound by any particular theory of operation, the inventive formulations surprisingly can form a depot of the detomidine or salt thereof following topical application to a subject's skin. As used herein, a “depot” represents an accumulation or a deposit of drug in a localized mass, from which the drug is gradually released to the surrounding tissue, thereby providing a prolonged, non-systemic effect for the treatment of pain. The present formulations can result in the formation of a depot of the detomidine or salt thereof in the subcutaneous tissue (hypodermis), the epidermis, or the dermis of a subject's skin. The formulations limit systemic exposure to the active agent and can achieve target engagement at the skin nociceptors in the epidermal skin layer. In certain embodiments, the depot includes crystals of the detomidine or salt thereof. Such crystals form within the subject's skin, including one or more of its layers, on which the formulation is topically applied, i.e., the crystals form following delivery of the detomidine or salt thereof into the subject's skin.

In accordance with these and other features, provided are topical formulations comprising about 0.001 to about 3 wt % detomidine or a salt thereof and, a carrier that is suitable for topical administration to a subject's skin, wherein the carrier optionally comprises a water-miscible solubilizer that is present in an amount of up to 40% by weight of the formulation, wherein the formulation has a pH of 4.5 to 9 and wherein said formulations are substantially free of 3-((1H-Imidazol-4-yl)methyl)-2-methylbenzaldehyde. Topical administration of the formulations to a subject can be effective to induce formation of a depot of detomidine or a salt thereof in the subject's skin and can be of use in providing prolonged, non-systemic treatment in a subject in need.

In accordance with these and other features, also provided are topical formulations comprising about 0.001 to about 3 wt % detomidine or a salt thereof and, a carrier that is suitable for topical administration to a subject's skin, wherein the carrier optionally comprises a water—miscible solubilizer that is present in an amount of up to 40% by weight of the formulation, wherein the formulation has a pH of 4.5 to 9 and wherein the formulation comprises less than about 1 wt % of 3-((1H-Imidazol-4-yl)methyl)-2-methylbenzaldehyde at least 6 months after its manufacture. Topical administration of the formulations to a subject can be effective to induce formation of a depot of detomidine or a salt thereof in the subject's skin and can be of use in providing prolonged, non-systemic treatment in a subject in need.

In accordance with these and other features, also provided are topical formulations comprising about 0.001 to about 3 wt % detomidine or a salt thereof and, a carrier that is suitable for topical administration to a subject's skin, wherein the carrier optionally comprises a water—miscible solubilizer that is present in an amount of up to 40% by weight of the formulation, wherein the formulation has a pH of 4.5 to 9 and wherein the topical formulation comprises less than 4% of the total detomidine content as crystalline detomidine base. Topical administration of the formulations to a subject can be effective to induce formation of a depot of detomidine or a salt thereof in the subject's skin and can be of use in providing prolonged, non-systemic treatment in a subject in need.

The formulations contain about 0.001 to about 3 wt % detomidine or a salt thereof. For example, the formulations may include about 0.005 to about 3, about 0.003 to about 3, about 0.008 to 3, about 0.01 to about 3, about 0.01 to about 2, about 0.01 to about 1.5, about 0.01 to about 1, 0.033 to about 1, 0.033 to about 0.33, about 0.05 to about 3, about 0.1 to about 3, about 0.1 to about 2.5, about 0.1 to about 2, about 0.1 to about 1.5, about 0.1 to about 1, about 0.33 to about 1, about 0.5 to about 1, or about 0.5 to about 0.75 wt % detomidine or salt thereof, or about 0.001, about 0.002, about 0.003, about 0.005, about 0.007, about 0.008, about 0.009, about 0.01, about 0.03, about 0.05, about 0.075, about 0.08, about 0.09, about 0.1, about 0.2, about 0.3, about 0.33, about 0.5, about 0.7, about 0.75, about 0.8, about 1.0, about 1.2, about 1.25, about 1.33, about 1.4, about 1.5, about 1.6, about 1.7, about 1.8, about 1.9, about 2.0, about 2.1, about 2.2, about 2.3, about 2.33, about 2.4, about 2.5, about 2.6, about 2.7, about 2.8, about 2.9, or about 3 wt % of detomidine or salt thereof.

The detomidine may be present in the formulations in the free base form or as a salt. Those of ordinary skill in the art can readily identify exemplary pharmaceutically acceptable salt forms of detomidine. Suitable pharmaceutically acceptable salts of detomidine include detomidine bitartrate, detomidine bitartrate hydrate, detomidine hydrochloride, detomidine p-toluenesulfonate, detomidine phosphate, detomidine thiosemicarbazone, detomidine sulfate, detomidine trifluoroacetate, detomidine hemipentahydrate, detomidine bitartrate hemipentahydrate, detomidine pentafluoropropionate, detomidine p-nitrophenylhydrazone, detomidine o-methyloxime, detomidine semicarbazone, detomidine hydrobromide, detomidine mucate, detomidine oleate, detomidine phosphate dibasic, detomidine phosphate monobasic, detomidine inorganic salt, detomidine organic salt, detomidine acetate trihydrate, detomidine bis(heptafluorobutyrate), detomidine bis(methylcarbamate), detomidine bis(pentafluoropropionate), detomidine bis(pyridine carboxylate), detomidine bis(trifluoroacetate), detomidine chlorhydrate, and detomidine sulfate pentahydrate. In certain embodiments of the presently disclosed dosage forms, the detomidine is present as the hydrochloride salt.

The formulations can also contain less than 1 wt % iso-detomidine, present in the free base form or as a salt. For example, the formulation can contain less than 1 wt %, less than 0.9 wt %, less than 0.8 wt %, less than 0.75 wt %, less than 0.7 wt %, less than 0.6 wt %, less than 0.5 wt %, less than 0.4 wt %, less than 0.3 wt %, less than 0.25 wt %, less than 0.2 wt %, less than 0.19 wt %, less than 0.18 wt %, less than 0.17 wt %, less than 0.16 wt %, less than 0.15 wt %, less than 0.14 wt %, less than 0.13 wt %, less than 0.12 wt %, less than 0.11 wt %, less than 0.1 wt %, less than 0.09 wt %, less than 0.08 wt %, less than 0.07 wt %, less than 0.06 wt %, less than 0.05 wt %, less than 0.04 wt %, less than 0.03 wt %, less than 0.02 wt %, less than 0.01 wt % or no identifiable iso-detomidine.

Accordingly, the formulations can contain less than about 1 wt % of 3-((1H-Imidazol-4-yl)methyl)-2-methylbenzaldehyde. For example, the formulation may contain less than 1 wt %, less than 0.9 wt %, less than 0.8 wt %, less than 0.75 wt %, less than 0.7 wt %, less than 0.6 wt %, less than 0.5 wt %, less than 0.4 wt %, less than 0.3 wt %, less than 0.25 wt %, less than 0.2 wt %, less than 0.19 wt %, less than 0.18 wt %, less than 0.17 wt %, less than 0.16 wt %, less than 0.15 wt %, less than 0.14 wt %, less than 0.13 wt %, less than 0.12 wt %, less than 0.11 wt %, less than 0.10 wt %, less than 0.09 wt %, less than 0.08 wt %, less than 0.07 wt %, less than 0.06 wt %, less than 0.05 wt %, less than 0.04 wt %, less than 0.03 wt %, less than 0.02 wt %, less than 0.01 wt % or no identifiable 3-((1H-Imidazol-4-yl)methyl)-2-methylbenzaldehyde at least 6 months, at least 9 months, at least 12 months, at least 18 months, at least 24 months, at least 30 months, at least 3 years, at least 4 years or at least 5 years after their manufacture. In certain embodiments, the formulations can contain less than about 1 wt % of 3-((1H-Imidazol-4-yl)methyl)-2-methylbenzaldehyde at least 6 months after their manufacture when stored at room temperature. In other embodiments, the formulations can contain less than about 1 wt % of 3-((1H-Imidazol-4-yl)methyl)-2-methylbenzaldehyde at least 6 months after their manufacture when stored in refrigerated conditions.

In certain embodiments, the formulations comprise less than about 1 wt % of 3-((1H-Imidazol-4-yl)methyl)-2-methylbenzaldehyde at least 6 months after their manufacture. In certain embodiments, the formulations comprise less than about 1 wt % of 3-((1H-Imidazol-4-yl)methyl)-2-methylbenzaldehyde at least 9 months after their manufacture. In certain embodiments, the formulations comprise less than about 1 wt % of 3-((1H-Imidazol-4-yl)methyl)-2-methylbenzaldehyde at least 12 months after their manufacture. In certain embodiments, the formulations comprise less than about 1 wt % of 3-((1H-Imidazol-4-yl)methyl)-2-methylbenzaldehyde at least 24 months after their manufacture. In certain embodiments, the formulations comprise less than about 0.5 wt % of 3-((1H-Imidazol-4-yl)methyl)-2-methylbenzaldehyde at least 6 months after their manufacture. In certain embodiments, the formulations comprise less than about 0.5 wt % of 3-((1H-Imidazol-4-yl)methyl)-2-methylbenzaldehyde at least 12 months after their manufacture. In certain embodiments, the formulations comprise less than about 1 wt % of 3-((1H-Imidazol-4-yl)methyl)-2-methylbenzaldehyde at least 24 months after their manufacture. In certain embodiments, the formulations are stored after their manufacture at room temperature for at least 12 months. In certain embodiments, the formulations are stored after their manufacture at room temperature for at least 24 months. In certain embodiments, the formulations are stored after their manufacture under refrigerated conditions for at least 12 months. In certain embodiments, the formulations are stored after their manufacture under refrigerated conditions for at least 24 months.

In certain embodiments, the formulations comprise less than about 0.2 wt % of 3-((1H-Imidazol-4-yl)methyl)-2-methylbenzaldehyde at least 6 months after their manufacture. In certain embodiments, the formulations comprise less than about 0.2 wt % of 3-((1H-Imidazol-4-yl)methyl)-2-methylbenzaldehyde at least 9 months after their manufacture. In certain embodiments, the formulations comprise less than about 0.2 wt % of 3-((1H-Imidazol-4-yl)methyl)-2-methylbenzaldehyde at least 12 months after their manufacture. In certain embodiments, the formulations comprise less than about 0.2 wt % of 3-((1H-Imidazol-4-yl)methyl)-2-methylbenzaldehyde at least 24 months after their manufacture. In certain embodiments, the formulations comprise less than about 0.19 wt % of 3-((1H-Imidazol-4-yl)methyl)-2-methylbenzaldehyde at least 6 months after their manufacture. In certain embodiments, the formulations comprise less than about 0.18 wt % of 3-((1H-Imidazol-4-yl)methyl)-2-methylbenzaldehyde at least 12 months after their manufacture. In certain embodiments, the formulations comprise less than about 0.18 wt % of 3-((1H-Imidazol-4-yl)methyl)-2-methylbenzaldehyde at least 24 months after their manufacture. In certain embodiments, the formulations are stored after their manufacture at room temperature for at least 12 months. In certain embodiments, the formulations are stored after their manufacture at room temperature for at least 24 months. In certain embodiments, the formulations are stored after their manufacture under refrigerated conditions for at least 12 months. In certain embodiments, the formulations are stored after their manufacture under refrigerated conditions for at least 24 months.

The formulations also include a carrier that is suitable for topical administration to a subject's skin. The carrier may include, for example, a solubilizer, a buffer, or both. As described below, the formulation may also include one or more additional components in order to produce the topical form, such as thickening or gelling agents, preservatives, antioxidants, permeation enhancers, emulsifying agents, emollients, or humectants.

Accordingly the formulations can include up to about 40% by weight of one or more solubilizers, such as a water-miscible solubilizer. The total amount of water-miscible solubilizer may be, for example, about 0.1 to about 40, about 0.1 to about 30, about 0.1 to about 20, about 0.1 to about 10, or about 0.1 to about 5% by weight, or about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4, about 5, about 6, about 7, about 8, about 9, about 10, about 12, about 14, about 16, about 18, about 20, about 22, about 25, about 27, about 30, about 32, about 35, about 37, or about 40 wt % in the present formulations. In some embodiments, the total amount of the solubilizer, e.g., water-miscible solubilizer, is not more than two times, not more than three times, or not more than four times the amount of the detomidine or salt thereof in the formulation.

Exemplary water-miscible solubilizers for the present formulations include alcohols, such as sugar alcohols, diols, polyols, or polyether alcohols, fatty acids, organic solvents, waxes, oils, poloxamers, cyclodextrins, or any combination thereof. For example, the solubilizer may be glycerol, polyethylene glycol (such as PEG 3350), propylene glycol, poloxamer 124, poloxamer 407, Labrasol® (caprylocaproyl polyoxyl-8 glycerides), Kleptose® HPB, Captisol® sulfobutylether β-cyclodextrin, or any combination thereof. In some embodiments, the water-miscible solubilizer is glycerol, propylene glycol, polyethylene glycol, or any combination thereof. For example, the water-miscible solubilizer may include both glycerol and propylene glycol.

The present formulations can also include a buffer that is effective to maintain the pH of the formulation at about 4.5 to about 9. For example, the buffer may be effective to maintain the pH at about 5.0 to about 9, about 5.0 to about 9, about 5.0 to about 8.5, about 5.0 to about 8.2, about 5.0 to about 8, about 5.0 to about 6.0, about 5.0 to about 5.5, about 5.2 to about 9, about 5.2 to about 8.5, about 5.2 to about 8.2, about 5.2 to about 8, about 5.2 to about 7, about 5.2 to about 6, about 5.2 to about 5.5, about 5.5 to about 9, about 5.5 to about 8.5, about 5.5 to about 8.2, or about 5.5 to about 8, or at about 4.5, about 4.6, about 4.7, about 4.8, about 4.9, about 5.0, about 5.1, about 5.2, about 5.3, about 5.4, about 5.5 about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8. about 7.9, about 8.0, about 8.1, about 8.2, about 8.3, about 8.4, about 8.5, about 8.6, about 8.7, about 8.8, about 8.9, or about 9.0. In certain embodiments, the pH of the formulation may be about 4.5 to about 7, for example about 4.5 to about 6, about 5 to about 6, or about 5.5. In certain embodiments, the pH of the formulation may be about 7 or lower, such as about 6 or lower. Buffers that may be used to maintain the pH of the formulation at a desired level can be readily identified by those of ordinary skill in the art, and may include, for example, water, phosphate buffer, sodium phosphate, buffer, Tris buffer, or citrate buffer. In the present formulations, the buffer may be present in a quantity that, when combined with the other components of the formulation, brings the total amount of components to 100 wt %.

The present formulations are designed for topical application to a subject's skin. Accordingly, the formulations are not configured for oral or transmucosal administration or for injection. Put differently, the formulations are non-oral, non-transmucosal and non-injectable.

The formulations can include a volatile solvent that at least partially evaporates from the skin surface following application. For example, in certain embodiments, the buffer component is aqueous, and the water that is contained within the aqueous buffer represents the volatile solvent. The portion of the formulation that remains following at least partial evaporation can be referred to as the “nonvolatile” or “residual” phase, and the volatile element(s) of the formulation that evaporate from the skin surface represents the “volatile” phase. In the present formulations, the detomidine or salt thereof is at or close to its saturation point within the nonvolatile phase following evaporation of the volatile phase. For example, the detomidine or salt thereof may be present in the residual phase following topical application of the instant formulations at or greater than about 75% of the saturation point of the active agent. In some embodiments, the detomidine or salt thereof is present in the residual phase at or greater than about 77, about 80, about 82, about 84, about 85, about 87, about 88, about 90, about 92, about 94, about 95, about 96, about 97, about 98, or about 99% of the saturation point for the detomidine or salt thereof.

In certain embodiments, the formulations may include an inert excipient that assists with increasing the concentration of the detomidine or salt thereof in the residual phase following topical application. In effect, such excipients can cause “salting out” of the detomidine or salt thereof from the other components of the residual phase, which can have the effect of increasing the activity of the detomidine or salt thereof on the surface of the subject's skin and promote permeability of the drug through the skin. Such inert excipients can include, for example, a polyol or simple sugar, such as sucrose, dextrose, trehalose, mannitol, sorbitol, or xylitol.

In certain embodiments, the formulations are designed to prevent the possible generation or increase in the amount of the aldehyde impurity, 3-((1H-Imidazol-4-yl)methyl)-2-methylbenzaldehyde, which has been identified as being generated and/or increasing in amount in an aqueous environment by oxidation processes. If not prevented by close control of manufacturing and packaging of exposure to both air and light, such generation and/or increases in the amount of the aldehyde impurity might thereby affect a water based formulation's long term stability and should therefore be avoided.

As noted above, and as described more fully below, the present formulations provide prolonged, substantially non-systemic treatment for pain and/or pruritus. The period of time over which the formulations can provide treatment is up to 24 hours when topically applied once a day. In certain embodiments, the formulations are preferably applied twice per day, and in such instances the treatment that is provided by a first of the two topical administrations has a duration that lasts until the second topical administration, and the second daily topical administration has a duration that lasts until the following day's first topical administration. As used herein, “substantially non-systemic” refers to a treatment effect that is localized to the bodily region (for example, body part) to which the formulation is topically applied, with a minimal or no medically relevant effect outside of that bodily region, or simply no minimal or no medically relevant systemic effect. Examples of minimal and/or medically relevant systemic effects can include sedation, decreased levels of consciousness, neurocognitive impairment, dizziness, hypotension, bradycardia and unsteady gait.

Being designed for topical application, the present formulations may take any appropriate form, including, for example, that of a cream, foam, gel, lotion, or ointment. As required and as described herein, the formulations according to the present disclosure may further comprise one or more additional components in order to produce the topical form, such as thickening or gelling agents, preservatives, antioxidants, permeation enhancers, emulsifying agents, emollients, or humectants.

Thickening or gelling agents can include, but are not limited to, hydroxymethyl cellulose, hydroxyethyl cellulose, hydroxypropyl cellulose, hydroxypropylmethyl cellulose, xanthan gum, carbomers (acrylates and acrylic acid and its derivatives polymers, such as Carbopol® 980 (crosslinked polyacrylate polymer)), povidones (e.g., polyvinylpyrrolidone), Poloxamers, Polyamide-3 (e.g., OleoCraft™ HP33), and other appropriate agents.

Preservatives can include, but are not limited to, benzalkonium chloride, parabens, sorbic acid and its salts, benzoic acid and its salts, cetrimonium bromide and chloride salts, phenoxyethanol, and other agents.

Antioxidants can include, but are not limited to, sodium metabisulfite, ascorbic acid, tocopheryl acetate (for purely aqueous formulations), and BHT or BHA (for hydrophobic formulations).

Permeation enhancers can include, but are not limited to, Transcutol® P (highly purified diethylene glycol monoethyl ether EP/NF) or dimethylisosorbide (DMI).

Emulsifying agents can include, but are not limited to, Tweens, Spans, poloxamers (124, 407, 188), Brij S2 and Brij 721, Crodex M (cetearyl alcohol and potassium cetyl Phosphate), Crodafos CES (cetearyl alcohol and dicetyl phosphate and Ceteth-10 phosphate (Crodafos CES), Cithrol DPHS (PEG 30 Dipolyhydroxystearate), cyclopentasiloxane, or macrogol hydroxystearate.

Emollients can include, but are not limited to, Migliol 810 or 812 (caprylic-capric triglycerides), Isoporpyl Isostearate (Crodamol IPIS), Isostearyl Isostearate (Crodamol ISIS), PPG-11 Stearyl Ether (Arlamol PS HE), Macrogol 6 Glycerol Caprylocaprate (Glycerox 767HC), or Labrasol® (caprylocaproyl polyoxyl-8 glycerides).

Humectants can include, but are not limited to, glycerin, propylene glycol, 1,3-propanediol, or 1,2-pentanediol.

The present formulations may contain detomidine or a salt thereof as the only therapeutic agent. In other embodiments, the formulations may include a further therapeutic agent in addition to the detomidine or a salt thereof. For example, the formulations may further comprise an analgesic (such as an NSAID, an opioid, or acetaminophen), an antidepressant agent (such as a tricyclic antidepressant), an anticonvulsant agent, a corticosteroid (such as prednisone or a derivative thereof), an antihistamine (such as a first, second or third generation H₁ antihistamine) or a local anesthetic (such as lidocaine, prilocaine, tetracaine, benzocaine, proxymetacaine, and the like). As another example, the formulations may further comprise one or more additional α-2-adrenergic agonists. Preferred α-2-adrenergic agonists include clonidine, romifidine, brimonidine, dexmedetomidine, and salts thereof.

In certain embodiments, the formulations comprise up to about 1 wt % of detomidine or salt thereof, and produce any one or more of: a blood plasma concentration of no more than about 500 pg/mL of the detomidine or salt thereof in the subject during the first 48 hours of twice-daily topical administration of said formulation; a blood plasma concentration of no more than about 500 pg/mL of the detomidine or salt thereof in the subject during the first 72 hours of twice-daily topical administration of the formulation; a blood plasma concentration of no more than about 500 pg/mL of the detomidine or salt thereof in the subject during the first 96 hours following topical application of the formulation on the subject; a blood plasma concentration of no more than about 800 pg/mL of the detomidine or salt thereof in the subject following the first 96 hours of twice-daily topical administration of the formulation; at least about 120 ng per mg of the subject's dermis per cm² of detomidine or salt thereof at any point during the first 24 hours of twice-daily topical administration of the formulation; at least about 180 ng per mg of the subject's dermis per cm² of detomidine or salt thereof at any point during the first 96 hours of twice-daily topical administration of the formulation; at least about 1200 ng per mg of the subject's epidermis per cm² of detomidine or salt thereof at any point during the first 24 hours of twice-daily topical administration of the formulation; at least about 4800 ng per mg of the subject's epidermis per cm² of detomidine or salt thereof at any point during the first 24 hours of twice-daily topical administration of the formulation; at least about 2000 ng per mg of the subject's epidermis per cm² of detomidine or salt thereof at any point during the first 96 hours and following the first 24 hours of twice-daily topical administration of the formulation; or, at least about 2400 ng per mg of the subject's epidermis per cm² of detomidine or salt thereof at any point during the first 96 hours and following the first 24 hours of twice-daily topical administration of the formulation.

Topical administration of the formulation once- or twice-daily to a subject for up to four days can result in a blood plasma concentration in the subject that is less than that required to achieve a systemic therapeutic effect of the detomidine or salt thereof.

In certain embodiments, the formulations are useful to provide prolonged, non-systemic treatment to a human subject when topically administered to the subject's skin.

In certain embodiments, the topical formulations comprise 0.01 to 5 wt % detomidine hydrochloride, glycerine, propylene glycol, a gelling agent, and a buffer that is effective to maintain the formulation at pH about 4.5 to about 8.2 and wherein said detomidine or a salt thereof, is substantially free of 3-((1H-Imidazol-4-yl)methyl)-2-methylbenzaldehyde.

In some embodiments, the topical formulations comprise 0.01 to 3 wt % detomidine hydrochloride, glycerine, propylene glycol, a cellulose gelling agent, and a buffer that is effective to maintain the formulation at pH about 4.5 to about 6 and wherein said detomidine or a salt thereof, is substantially free of 3-((1H-Imidazol-4-yl)methyl)-2-methylbenzaldehyde.

In some other embodiments, the topical formulations comprise 0.05 to 3 wt % detomidine hydrochloride, glycerine, propylene glycol, a cellulose gelling agent, and a buffer that is effective to maintain the formulation at pH about 5 to about 6 and wherein said detomidine or a salt thereof, is substantially free of 3-((1H-Imidazol-4-yl)methyl)-2-methylbenzaldehyde.

In yet other embodiments, the topical formulations comprise 0.1 to 2 wt % detomidine hydrochloride, glycerine, propylene glycol, a cellulose gelling agent, and a buffer that is effective to maintain the formulation at pH about 5 to about 5.5 and wherein said detomidine or a salt thereof, is substantially free of 3-((1H-Imidazol-4-yl)methyl)-2-methylbenzaldehyde.

In still other embodiments, the topical formulations comprise 0.1 to 1 wt % detomidine hydrochloride, glycerine, propylene glycol, hydroxy ethyl cellulose, and a buffer that is effective to maintain the formulation at pH about 5 to about 5.5 and wherein said detomidine or a salt thereof, is substantially free of 3-((1H-Imidazol-4-yl)methyl)-2-methylbenzaldehyde.

In yet other embodiments, the topical formulations comprise 0.1 to 1 wt % detomidine hydrochloride, glycerine, propylene glycol, hydroxy ethyl cellulose, and a buffer that is effective to maintain the formulation at pH about 5.2 to about 5.5 and wherein said detomidine or a salt thereof, is substantially free of 3-((1H-Imidazol-4-yl)methyl)-2-methylbenzaldehyde.

In certain embodiments, the topical formulations comprise 0.01 to 5 wt % detomidine hydrochloride, glycerine, propylene glycol, a gelling agent, and a buffer that is effective to maintain the formulation at pH about 4.5 to about 8.2 and wherein the formulation comprises less than about 1 wt % of 3-((1H-Imidazol-4-yl)methyl)-2-methylbenzaldehyde at least 6 months after its manufacture.

In some embodiments, the topical formulations comprise 0.01 to 3 wt % detomidine hydrochloride, glycerine, propylene glycol, a cellulose gelling agent, and a buffer that is effective to maintain the formulation at pH about 4.5 to about 6 and wherein the formulation comprises less than about 1 wt % of 3-((1H-Imidazol-4-yl)methyl)-2-methylbenzaldehyde at least 6 months after its manufacture.

In some other embodiments, the topical formulations comprise 0.05 to 3 wt % detomidine hydrochloride, glycerine, propylene glycol, a cellulose gelling agent, and a buffer that is effective to maintain the formulation at pH about 5 to about 6 and wherein the formulation comprises less than about 1 wt % of 3-((1H-Imidazol-4-yl)methyl)-2-methylbenzaldehyde at least 6 months after its manufacture.

In yet other embodiments, the topical formulations comprise 0.1 to 2 wt % detomidine hydrochloride, glycerine, propylene glycol, a cellulose gelling agent, and a buffer that is effective to maintain the formulation at pH about 5 to about 5.5 and wherein the formulation comprises less than about 1 wt % of 3-((1H-Imidazol-4-yl)methyl)-2-methylbenzaldehyde at least 6 months after its manufacture.

In still other embodiments, the topical formulations comprise 0.1 to 1 wt % detomidine hydrochloride, glycerine, propylene glycol, hydroxy ethyl cellulose, and a buffer that is effective to maintain the formulation at pH about 5 to about 5.5 and wherein the formulation comprises less than about 1 wt % of 3-((1H-Imidazol-4-yl)methyl)-2-methylbenzaldehyde at least 6 months after its manufacture.

In yet other embodiments, the topical formulations comprise 0.1 to 1 wt % detomidine hydrochloride, glycerine, propylene glycol, hydroxy ethyl cellulose, and a buffer that is effective to maintain the formulation at pH about 5.2 to about 5.5 and wherein the formulation comprises less than about 1 wt % of 3-((1H-Imidazol-4-yl)methyl)-2-methylbenzaldehyde at least 6 months after its manufacture.

In certain embodiments, the topical formulations comprise 0.01 to 5 wt % detomidine hydrochloride, glycerine, propylene glycol, a gelling agent, and a buffer that is effective to maintain the formulation at pH about 4.5 to about 8.2 and wherein the formulation comprises less than about 0.2 wt % of 3-((1H-Imidazol-4-yl)methyl)-2-methylbenzaldehyde at least 6 months after its manufacture.

In some embodiments, the topical formulations comprise 0.01 to 3 wt % detomidine hydrochloride, glycerine, propylene glycol, a cellulose gelling agent, and a buffer that is effective to maintain the formulation at pH about 4.5 to about 6 and wherein the formulation comprises less than about 0.2 wt % of 3-((1H-Imidazol-4-yl)methyl)-2-methylbenzaldehyde at least 6 months after its manufacture.

In some other embodiments, the topical formulations comprise 0.05 to 3 wt % detomidine hydrochloride, glycerine, propylene glycol, a cellulose gelling agent, and a buffer that is effective to maintain the formulation at pH about 5 to about 6 and wherein the formulation comprises less than about 0.2 wt % of 3-((1H-Imidazol-4-yl)methyl)-2-methylbenzaldehyde at least 6 months after its manufacture.

In yet other embodiments, the topical formulations comprise 0.1 to 2 wt % detomidine hydrochloride, glycerine, propylene glycol, a cellulose gelling agent, and a buffer that is effective to maintain the formulation at pH about 5 to about 5.5 and wherein the formulation comprises less than about 0.2 wt % of 3-((1H-Imidazol-4-yl)methyl)-2-methylbenzaldehyde at least 6 months after its manufacture.

In still other embodiments, the topical formulations comprise 0.1 to 1 wt % detomidine hydrochloride, glycerine, propylene glycol, hydroxy ethyl cellulose, and a buffer that is effective to maintain the formulation at pH about 5 to about 5.5 and wherein the formulation comprises less than about 0.2 wt % of 3-((1H-Imidazol-4-yl)methyl)-2-methylbenzaldehyde at least 6 months after its manufacture.

In yet other embodiments, the topical formulations comprise 0.1 to 1 wt % detomidine hydrochloride, glycerine, propylene glycol, hydroxy ethyl cellulose, and a buffer that is effective to maintain the formulation at pH about 5.2 to about 5.5 and wherein the formulation comprises less than about 0.2 wt % of 3-((1H-Imidazol-4-yl)methyl)-2-methylbenzaldehyde at least 6 months after its manufacture.

Any of embodiment of the invention may further comprise a preservative. Furthermore, any of these embodiments may be effective to produce a depot of the detomidine or salt thereof in the subject's skin following topical application.

The present disclosure also pertains to topical formulations comprising about 0.001 to about 3 wt % detomidine or a salt thereof, a carrier suitable for topical application to the skin of a subject, wherein the formulation is effective to provide relief from pain and/or pruritus in the subject while producing a maximum blood plasma concentration of no more than about 1600 pg/mL after four days of twice daily administration of said formulation and wherein said detomidine or a salt thereof, is substantially free of 3-((1H-Imidazol-4-yl)methyl)-2-methylbenzaldehyde.

In certain embodiments, the topical formulations comprise 0.01 to 1 wt % detomidine hydrochloride, a gelling agent, a water-miscible solubilizer, and a buffer that is effective to provide a pH of about 4.5 to about 7 and wherein said detomidine or a salt thereof, is substantially free of 3-((1H-Imidazol-4-yl)methyl)-2-methylbenzaldehyde.

In certain embodiments, the topical formulations comprise 0.01 to 1 wt % detomidine hydrochloride, a gelling agent, a water-miscible solubilizer, and a buffer that is effective to provide a pH of about 5 to about 6 and wherein said detomidine or a salt thereof, is substantially free of 3-((1H-Imidazol-4-yl)methyl)-2-methylbenzaldehyde.

In certain embodiments, the topical formulations comprise 0.01 to 0.5 wt % detomidine hydrochloride, a gelling agent, a water-miscible solubilizer, and a buffer that is effective to provide a pH of about 4.5 to about 7 and wherein said detomidine or a salt thereof, is substantially free of 3-((1H-Imidazol-4-yl)methyl)-2-methylbenzaldehyde.

In certain embodiments, the topical formulations comprise 0.01 to 0.5 wt % detomidine hydrochloride, a gelling agent, a water-miscible solubilizer, and a buffer that is effective to provide a pH of about 5 to about 6 and wherein said detomidine or a salt thereof, is substantially free of 3-((1H-Imidazol-4-yl)methyl)-2-methylbenzaldehyde.

In certain embodiments, the topical formulations comprise 0.01 to 0.5 wt % detomidine hydrochloride, a gelling agent, a water-miscible solubilizer, and a buffer that is effective to provide a pH of about 5.5 and wherein said detomidine or a salt thereof, is substantially free of 3-((1H-Imidazol-4-yl)methyl)-2-methylbenzaldehyde.

In certain embodiments, the topical formulations comprise 0.03 to 0.2 wt % detomidine hydrochloride, a gelling agent, a water-miscible solubilizer, and a buffer that is effective to provide a pH of about 4.5 to about 7 and wherein said detomidine or a salt thereof, is substantially free of 3-((1H-Imidazol-4-yl)methyl)-2-methylbenzaldehyde.

In certain embodiments, the topical formulations comprise 0.03 to 0.2 wt % detomidine hydrochloride, a gelling agent, a water-miscible solubilizer, and a buffer that is effective to provide a pH of about 5 to about 6 and wherein said detomidine or a salt thereof, is substantially free of 3-((1H-Imidazol-4-yl)methyl)-2-methylbenzaldehyde.

In certain embodiments, the topical formulations comprise 0.05 to 0.15 wt % detomidine hydrochloride, a gelling agent, a water-miscible solubilizer, and a buffer that is effective to provide a pH of about 4.5 to about 7 and wherein said detomidine or a salt thereof, is substantially free of 3-((1H-Imidazol-4-yl)methyl)-2-methylbenzaldehyde.

In certain embodiments, the topical formulations comprise 0.05 to 0.15 wt % detomidine hydrochloride, a gelling agent, a water-miscible solubilizer, and a buffer that is effective to provide a pH of about 5 to about 6 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, a gelling agent, a water-miscible solubilizer, and a buffer that is effective to provide a pH of about 4.5 to about 7 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, a gelling agent, a water-miscible solubilizer, and a buffer that is effective to provide a pH of about 5 to about 6 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, a gelling agent, a water-miscible solubilizer, and a buffer that is effective to provide a pH of about 5.5 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, about 1% to about 3% of a gelling agent, about 0.1% to about 50% of a water-miscible solubilizer, and a buffer that is effective to provide a pH of about 4.5 to about 7 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, about 1% to about 3% of a gelling agent, about 0.1% to about 30% of a water-miscible solubilizer, and a buffer that is effective to provide a pH of about 4.5 to about 7 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, about 1% to about 3% of a gelling agent, about 0.1% to about 30% of a water-miscible solubilizer, and a buffer that is effective to provide a pH of about 5 to about 6 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, about 1% to about 3% of a gelling agent, about 0.1% to about 10% of a water-miscible solubilizer, and a buffer that is effective to provide a pH of about 4.5 to about 7 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, about 1% to about 3% of a gelling agent, about 0.1% to about 1% of a water-miscible solubilizer, and a buffer that is effective to provide a pH of about 4.5 to about 7 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, about 1% to about 3% of a gelling agent, about 0.1% to about 1% of a water-miscible solubilizer, and a buffer that is effective to provide a pH of about 5 to about 7 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, about 1% to about 3% of a gelling agent, about 0.1% to about 1% of a water-miscible solubilizer, and a buffer that is effective to provide a pH of about 5 to about 6 and wherein said detomidine or a salt thereof, is substantially free of 3-((1H-Imidazol-4-yl)methyl)-2-methylbenzaldehyde.

In certain embodiments, the topical formulations comprise 0.01 to 3 wt % detomidine hydrochloride, a gelling agent, a water-miscible solubilizer, and a buffer that is effective to provide a pH of about 5.0 to about 5.5 and wherein said detomidine or a salt thereof, is substantially free of 3-((1H-Imidazol-4-yl)methyl)-2-methylbenzaldehyde.

In certain embodiments, the topical formulations comprise 0.01 to 3 wt % detomidine hydrochloride, a gelling agent, a water-miscible solubilizer, and a buffer that is effective to provide a pH of about 5.2 to about 5.5 and wherein said detomidine or a salt thereof, is substantially free of 3-((1H-Imidazol-4-yl)methyl)-2-methylbenzaldehyde.

In certain embodiments, the topical formulations comprise 0.01 to 1 wt % detomidine hydrochloride, a gelling agent, a water-miscible solubilizer, and a buffer that is effective to provide a pH of about 4.5 to about 7 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise 0.01 to 0.5 wt % detomidine hydrochloride, a gelling agent, a water-miscible solubilizer, and a buffer that is effective to provide a pH of about 4.5 to about 7 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise 0.01 to 0.5 wt % detomidine hydrochloride, a gelling agent, a water-miscible solubilizer, and a buffer that is effective to provide a pH of about 5.5 and wherein the formulation comprises less than about 1 wt % of 3-((1H-Imidazol-4-yl)methyl)-2-methylbenzaldehyde at least 6 months after its manufacture.

In certain embodiments, the topical formulations comprise 0.03 to 1 wt % detomidine hydrochloride, a gelling agent, a water-miscible solubilizer, and a buffer that is effective to provide a pH of about 4.5 to about 7 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise 0.05 to 0.15 wt % detomidine hydrochloride, a gelling agent, a water-miscible solubilizer, and a buffer that is effective to provide a pH of about 4.5 to about 7 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise about 0.1 wt % detomidine hydrochloride, a gelling agent, a water-miscible solubilizer, and a buffer that is effective to provide a pH of about 4.5 to about 7 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise about 0.1 wt % detomidine hydrochloride, a gelling agent, a water-miscible solubilizer, and a buffer that is effective to provide a pH of about 5.5 and wherein the formulation comprises less than about 1 wt % of 3-((1H-Imidazol-4-yl)methyl)-2-methylbenzaldehyde at least 6 months after its manufacture.

In certain embodiments, the topical formulations comprise about 0.33 wt % detomidine hydrochloride, a gelling agent, a water-miscible solubilizer, and a buffer that is effective to provide a pH of about 4.5 to about 7 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise about 0.33 wt % detomidine hydrochloride, a gelling agent, a water-miscible solubilizer, and a buffer that is effective to provide a pH of about 5.5 and wherein the formulation comprises less than about 1 wt % of 3-((1H-Imidazol-4-yl)methyl)-2-methylbenzaldehyde at least 6 months after its manufacture.

In certain embodiments, the topical formulations comprise about 1 wt % detomidine hydrochloride, a gelling agent, a water-miscible solubilizer, and a buffer that is effective to provide a pH of about 4.5 to about 7 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise about 1 wt % detomidine hydrochloride, a gelling agent, a water-miscible solubilizer, and a buffer that is effective to provide a pH of about 5.5 and wherein the formulation comprises less than about 1 wt % of 3-((1H-Imidazol-4-yl)methyl)-2-methylbenzaldehyde at least 6 months after its manufacture.

In certain embodiments, the topical formulations comprise about 0.1 wt % detomidine hydrochloride, about 1% to about 3% of a gelling agent, about 0.1% to about 50% of a water-miscible solubilizer, and a buffer that is effective to provide a pH of about 4.5 to about 7 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7. In certain embodiments, the formulation comprises about 0.1% to about 50% of a water-miscible solubilizer. In certain embodiments, the formulation comprises about 0.1% to about 30% of a water-miscible solubilizer. In certain embodiments, the formulation comprises about 0.1% to about 10% of a water-miscible solubilizer. In certain embodiments, the formulation comprises about 0.1% to about 1% of a water-miscible solubilizer. In certain embodiments, the topical formulations comprise about 0.1 wt % detomidine hydrochloride, about 1% to about 3% of a gelling agent, about 0.1% to about 50% of a water-miscible solubilizer, and a buffer that is effective to provide a pH of about 4.5 to about 7 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7. In certain embodiments, the formulation comprises about 0.1% to about 50% of a water-miscible solubilizer. In certain embodiments, the formulation comprises about 0.1% to about 30% of a water-miscible solubilizer. In certain embodiments, the formulation comprises about 0.1% to about 10% of a water-miscible solubilizer. In certain embodiments, the formulation comprises about 0.1% to about 1% of a water-miscible solubilizer.

In certain embodiments, the topical formulations comprise about 0.33 wt % detomidine hydrochloride, about 1% to about 3% of a gelling agent, about 0.1% to about 50% of a water-miscible solubilizer, and a buffer that is effective to provide a pH of about 4.5 to about 7 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7. In certain embodiments, the formulation comprises about 0.1% to about 50% of a water-miscible solubilizer. In certain embodiments, the formulation comprises about 0.1% to about 30% of a water-miscible solubilizer. In certain embodiments, the formulation comprises about 0.1% to about 10% of a water-miscible solubilizer. In certain embodiments, the formulation comprises about 0.1% to about 1% of a water-miscible solubilizer.

In certain embodiments, the topical formulations comprise about 1 wt % detomidine hydrochloride, about 1% to about 3% of a gelling agent, about 0.1% to about 50% of a water-miscible solubilizer, and a buffer that is effective to provide a pH of about 4.5 to about 7 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7. In certain embodiments, the formulation comprises about 0.1% to about 50% of a water-miscible solubilizer. In certain embodiments, the formulation comprises about 0.1% to about 30% of a water-miscible solubilizer. In certain embodiments, the formulation comprises about 0.1% to about 10% of a water-miscible solubilizer. In certain embodiments, the formulation comprises about 0.1% to about 1% of a water-miscible solubilizer. In certain embodiments, the topical formulations comprise 0.01 to 1 wt % detomidine hydrochloride, a gelling agent, a water-miscible solubilizer, and a buffer that is effective to provide a pH of about 4.5 to about 7 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise 0.01 to 0.5 wt % detomidine hydrochloride, a gelling agent, a water-miscible solubilizer, and a buffer that is effective to provide a pH of about 4.5 to about 7 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise 0.01 to 0.5 wt % detomidine hydrochloride, a gelling agent, a water-miscible solubilizer, and a buffer that is effective to provide a pH of about 5.5 and wherein the formulation comprises less than about 0.2 wt % of 3-((1H-Imidazol-4-yl)methyl)-2-methylbenzaldehyde at least 6 months after its manufacture.

In certain embodiments, the topical formulations comprise 0.03 to 0.2 wt % detomidine hydrochloride, a gelling agent, a water-miscible solubilizer, and a buffer that is effective to provide a pH of about 4.5 to about 7 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise 0.05 to 0.15 wt % detomidine hydrochloride, a gelling agent, a water-miscible solubilizer, and a buffer that is effective to provide a pH of about 4.5 to about 7 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise about 0.1 wt % detomidine hydrochloride, a gelling agent, a water-miscible solubilizer, and a buffer that is effective to provide a pH of about 4.5 to about 7 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise about 0.1 wt % detomidine hydrochloride, a gelling agent, a water-miscible solubilizer, and a buffer that is effective to provide a pH of about 5.5 and wherein the formulation comprises less than about 0.2 wt % of 3-((1H-Imidazol-4-yl)methyl)-2-methylbenzaldehyde at least 6 months after its manufacture.

In certain embodiments, the topical formulations comprise about 0.33 wt % detomidine hydrochloride, a gelling agent, a water-miscible solubilizer, and a buffer that is effective to provide a pH of about 4.5 to about 7 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise about 0.33 wt % detomidine hydrochloride, a gelling agent, a water-miscible solubilizer, and a buffer that is effective to provide a pH of about 5.5 and wherein the formulation comprises less than about 0.2 wt % of 3-((1H-Imidazol-4-yl)methyl)-2-methylbenzaldehyde at least 6 months after its manufacture.

In certain embodiments, the topical formulations comprise about 1 wt % detomidine hydrochloride, a gelling agent, a water-miscible solubilizer, and a buffer that is effective to provide a pH of about 4.5 to about 7 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 6.

In certain embodiments, the topical formulations comprise about 1 wt % detomidine hydrochloride, a gelling agent, a water-miscible solubilizer, and a buffer that is effective to provide a pH of about 5.5 and wherein the formulation comprises less than about 0.2 wt % of 3-((1H-Imidazol-4-yl)methyl)-2-methylbenzaldehyde at least 6 months after its manufacture.

In certain embodiments, the topical formulations comprise about 0.1 wt % detomidine hydrochloride, about 1% to about 3% of a gelling agent, about 0.1% to about 50% of a water-miscible solubilizer, and a buffer that is effective to provide a pH of about 4.5 to about 7 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7. In certain embodiments, the formulation comprises about 0.1% to about 50% of a water-miscible solubilizer. In certain embodiments, the formulation comprises about 0.1% to about 30% of a water-miscible solubilizer. In certain embodiments, the formulation comprises about 0.1% to about 10% of a water-miscible solubilizer. In certain embodiments, the formulation comprises about 0.1% to about 1% of a water-miscible solubilizer. In certain embodiments, the topical formulations comprise about 0.1 wt % detomidine hydrochloride, about 1% to about 3% of a gelling agent, about 0.1% to about 50% of a water-miscible solubilizer, and a buffer that is effective to provide a pH of about 4.5 to about 7 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7. In certain embodiments, the formulation comprises about 0.1% to about 50% of a water-miscible solubilizer. In certain embodiments, the formulation comprises about 0.1% to about 30% of a water-miscible solubilizer. In certain embodiments, the formulation comprises about 0.1% to about 10% of a water-miscible solubilizer. In certain embodiments, the formulation comprises about 0.1% to about 1% of a water-miscible solubilizer.

In certain embodiments, the topical formulations comprise about 0.33 wt % detomidine hydrochloride, about 1% to about 3% of a gelling agent, about 0.1% to about 50% of a water-miscible solubilizer, and a buffer that is effective to provide a pH of about 4.5 to about 7 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7. In certain embodiments, the formulation comprises about 0.1% to about 50% of a water-miscible solubilizer. In certain embodiments, the formulation comprises about 0.1% to about 30% of a water-miscible solubilizer. In certain embodiments, the formulation comprises about 0.1% to about 10% of a water-miscible solubilizer. In certain embodiments, the formulation comprises about 0.1% to about 1% of a water-miscible solubilizer.

In certain embodiments, the topical formulations comprise about 1 wt % detomidine hydrochloride, about 1% to about 3% of a gelling agent, about 0.1% to about 50% of a water-miscible solubilizer, and a buffer that is effective to provide a pH of about 4.5 to about 7 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7. In certain embodiments, the formulation comprises about 0.1% to about 50% of a water-miscible solubilizer. In certain embodiments, the formulation comprises about 0.1% to about 30% of a water-miscible solubilizer. In certain embodiments, the formulation comprises about 0.1% to about 10% of a water-miscible solubilizer. In certain embodiments, the formulation comprises about 0.1% to about 1% of a water-miscible solubilizer.

In certain embodiments, the topical formulations comprise about 0.1 wt % detomidine hydrochloride, about 1% to about 3% of a gelling agent, about 0.1% to about 1% of a water-miscible solubilizer, and a buffer that is effective to provide a pH of about 5.5.

In certain embodiments, the topical formulations comprise about 0.1 wt % detomidine hydrochloride, about 1% to about 3% of hydroxy ethyl cellulose, about 0.1% to about 1% of glycerin, and a buffer that is effective to provide a pH of about 5 to about 6 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, about 1% to about 3% of hydroxy ethyl cellulose, about 0.1% to about 1% of glycerin, and a buffer that is effective to provide a pH of about 5.5 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, about 1.75% of hydroxy ethyl cellulose, about 0.3% of glycerin, and a buffer that is effective to provide a pH of about 5 to about 6 and wherein said detomidine or a salt thereof, is substantially free of 3-((1H-Imidazol-4-yl)methyl)-2-methylbenzaldehyde.

In certain embodiments, the topical formulations comprise about 0.1 wt % detomidine hydrochloride, about 1.75% of hydroxy ethyl cellulose, about 0.3% of glycerin, and a buffer that is effective to provide a pH of about 5.5 and wherein said detomidine or a salt thereof, is substantially free of 3-((1H-Imidazol-4-yl)methyl)-2-methylbenzaldehyde. In certain embodiments, the topical formulations comprise about 0.1 wt % detomidine hydrochloride, about 1% to about 3% of hydroxy ethyl cellulose, about 0.1% to about 1% of glycerin, and a buffer that is effective to provide a pH of about 5 to about 6 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7.

In certain embodiments, the topical formulations comprise about 0.33 wt % detomidine hydrochloride, about 1% to about 3% of hydroxy ethyl cellulose, about 0.1% to about 1% of glycerin, and a buffer that is effective to provide a pH of about 5 to about 6 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7.

In certain embodiments, the topical formulations comprise about 1 wt % detomidine hydrochloride, about 1% to about 3% of hydroxy ethyl cellulose, about 0.1% to about 1% of glycerin, and a buffer that is effective to provide a pH of about 5 to about 6 and wherein the formulation comprises less than about 1 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7.

In certain embodiments, the topical formulations comprise about 0.1 wt % detomidine hydrochloride, about 1% to about 3% of hydroxy ethyl cellulose, about 0.1% to about 1% of glycerin, and a buffer that is effective to provide a pH of about 5 to about 6 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7.

In certain embodiments, the topical formulations comprise about 0.33 wt % detomidine hydrochloride, about 1% to about 3% of hydroxy ethyl cellulose, about 0.1% to about 1% of glycerin, and a buffer that is effective to provide a pH of about 5 to about 6 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7.

In certain embodiments, the topical formulations comprise about 1 wt % detomidine hydrochloride, about 1% to about 3% of hydroxy ethyl cellulose, about 0.1% to about 1% of glycerin, and a buffer that is effective to provide a pH of about 5 to about 6 and wherein the formulation comprises less than about 0.2 wt % of 3-((1H-Imidazol-4-yl)methyl)-2-methylbenzaldehyde at least 6 months after its manufacture. In certain embodiments, the buffer is effective to provide a pH of about 4.5 to about 7. In certain embodiments, the buffer is effective to provide a pH of about 5 to about 5.5. In certain embodiments, the buffer is effective to provide a pH of about 5.2 to about 5.5. In certain embodiments, the buffer is effective to provide a pH of about 5 to about 6. In certain embodiments, the buffer is effective to provide a pH of about 5 to about 7

The carrier component of the present formulations can comprise one or both of a water-miscible solubilizer and a buffer. Unless otherwise specified below, such formulations may share any one or more of the characteristics described above with respect to the other inventive formulations, including the quantity of the detomidine or salt thereof; the identity of possible salt forms of the detomidine; the presence, and, where present, the amount and type of possible water-miscible solubilizers; the pH of the formulation; the identity of possible buffers for maintaining the desired pH; the percentage of detomidine or salt thereof relative to its saturation point in the residual phase of the formulation following application to the skin; the presence and identity of optional additional therapeutic agents; the presence of an inert excipient that assists with increasing the concentration of the detomidine or salt thereof in the residual phase following topical application; and, the presence and identity of optional additional components other than a further therapeutic agent or inert excipient. Thus, the detailed description of these characteristics provided above can apply to the present formulations, as well.

In certain embodiments, the formulations comprise up to 1 wt % of detomidine or salt thereof, and produce any one or more of: a maximum blood plasma concentration of no more than about 1400 pg/mL after three days of twice daily administration of the formulation; a maximum blood plasma concentration of no more than about 1200 pg/mL after two days of twice daily administration of the formulation; a maximum blood plasma concentration of no more than about 800 pg/mL after the first day of twice daily administration of the formulation; at least about 120 ng per mg of the subject's dermis per cm² of detomidine or salt thereof at any point during the first 24 hours of twice daily topical administration of the formulation; at least about 180 ng per mg of the subject's dermis per cm² of detomidine or salt thereof at any point during the first 96 hours of twice-daily topical administration of the formulation; at least about 1200 ng per mg of the subject's epidermis per cm² of detomidine or salt thereof at any point during the first 24 hours of twice-daily topical administration of the formulation; at least about 4800 ng per mg of the subject's epidermis per cm² of detomidine or salt thereof at any point during the first 24 hours of twice-daily topical administration of the formulation; at least about 2000 ng per mg of the subject's epidermis per cm² of detomidine or salt thereof at any point during the first 96 hours and following the first 24 hours of twice-daily topical administration of the formulation; or, at least about 2400 ng per mg of the subject's epidermis per cm² of detomidine or salt thereof at any point during the first 96 hours and following the first 24 hours of twice-daily topical administration of the formulation.

Topical administration of the formulations once- or twice-daily to a subject for up to four days can result in a blood plasma concentration in the subject that is less than that required to achieve a systemic therapeutic effect of the detomidine or salt thereof. According to one embodiment, topical administration of the formulations once- or twice-daily to a subject for at least four days results in a blood plasma concentration in the subject that remains less than that required to achieve a systemic therapeutic effect of the detomidine or salt thereof. Preferably, the topical administration can continue for weeks, months, or longer while maintaining a sub-therapeutic systemic blood plasma concentration.

The present invention also provides methods for treating pain in a subject in need thereof comprising topically administering to the subject an effective amount of a formulation according to any one of the embodiments disclosed herein. The present disclosure also provides methods for providing prolonged, non-systemic treatment for pain in a subject in need thereof comprising topically administering to the subject a formulation according to any one of the embodiments disclosed above. In certain embodiments, the pain treated according to the methods of the present invention is neuropathic pain. In certain embodiments, the neuropathic pain is postherpetic neuralgia. In certain embodiments, the neuropathic pain is diabetic peripheral neuropathy.

The pain for which any of the above methods provide treatment can be any type of pain for which topical treatment is relevant, whether acute or chronic. For example, the pain for which treatment is provided using the present methods may be somatic (caused by the activation of pain receptors in either the body surface or musculoskeletal tissues, such as postsurgical pain), visceral (caused by damage or injury to internal body structures or organs), or neuropathic (post-herpetic neuralgia and diabetic neuropathy representing examples). Exemplary types of pain that can be treated according to the present methods include carpal tunnel syndrome, abdominal pain, hip pain, knee and other joint pain, pain deriving from piriformis syndrome, back pain, neck or shoulder pain, acute or chronic muscle pain, trigeminal neuralgia, post-herpetic neuralgia, sciatica pain, arachnoiditis (spinal pain), pain from complex regional pain syndrome, phantom limb pain diabetes-related nerve pain (neuropathy), pain deriving from depression or anxiety, and pain from compartment syndrome.

The present invention also provides methods for treating pruritus in a subject in need thereof comprising topically administering to the subject an effective amount of a formulation according to any one of the embodiments disclosed herein. The present disclosure also provides methods for providing prolonged, non-systemic treatment for pruritus in a subject in need thereof comprising topically administering to the subject a formulation according to any one of the embodiments disclosed above. In certain embodiments, the pruritus treated according to the methods of the present invention is acute pruritus, namely pruritus that has manifested for up to six consecutive weeks. In certain embodiments, the pruritus treated according to the methods of the present invention is chronic pruritus, namely pruritus that has manifested for more than six consecutive weeks.

The pruritus for which any of the above methods provide treatment can be any type of pain for which topical treatment is relevant, whether acute or chronic. For example, the pruritus for which treatment is provided using the present methods may be a symptom of a dermatological, neurological, psychogenic or systemic condition, or it may be of mixed origin.

Examples of pruritic dermatological conditions include atopic dermatitis, contact dermatitis, allergic dermatitis, seborrheic dermatitis, statis dermatitis, psoriasis, parapsoriasis, pityriasis rubra pilaris, pityriasis rosea, acne, dermatitis herpetiformis, bullous pemphigoid, lichen planus, prurigo nodularis, lichen simplex chronicus, lichen amyloidosis, urticaria, mastocytosis, polymorphous light eruption, actinic prurigo, actinic dermatitis, polymorphic eruption of pregnancy, eosinophilic folliculitis, dermatomyositis, prurigo pigmentosa, lichen sclerosus, palmoplantar pustulosis, pompholyx, idiopathic xerosis, scarring, burns, burn scars, keloid scars, hypertrophic scars, reactive drug eruptions and pruritus of an infestive or infective origin. Examples of pruritic infestive conditions include scabies, pediculosis and arthropod bites. Examples of pruritic infective conditions include fungal, parasitic, viral and bacterial conditions.

Examples of pruritic neurological conditions include notalgia paresthetica, brachioradial pruritus, post-herpetic neuralgia, stroke, small fiber neuropathy, trigeminal trophic syndrome, Creutzfeldt-Jakob disease, chemotherapy-induced neuropathy, HIV-related neuropathy and multiple sclerosis.

Examples of pruritic psychogenic conditions include depression, anxiety, psychogenic excoriation, anorexia nervosa and delusional parasitosis.

Examples of pruritic systemic conditions include chronic renal failure, uremic pruritus, liver disease, primary biliary cholangitis, primary biliary cirrhosis, cholestatic jaundice, hepatitis C, cholestasis of pregnancy, polycythemia vera, iron deficiency anemia, Hodgkin lymphoma, non-Hodgkin lymphoma, multiple myeloma, hematologic or lymphoproliferative disorders, primary cutaneous lymphoma, mycosis fungoides, cutaneous T cell lymphoma, malignancy, plasma cell dyscrasias, gastric carcinoid tumors, hyperthyroidism, hypothyroidism, hyperparathyroidism, haemochromatosis, celiac disease, systemic lupus erythematosus, systemic sclerosis, diabetes, carcinoid syndrome, dermatomyositis, scleroderma, Sjagren syndrome, linear immunoglobulin A (IgA) disease, graft-versus-host disease, Darier disease, Hailey-Hailey disease, porphyria and amyloidosis.

Also provided herein are methods for forming a subcutaneous (i.e., below skin surface) depot of detomidine or a salt thereof in a subject comprising topically administering to the subject a formulation according to any one of the above-described embodiments, wherein the detomidine or salt thereof is released from the depot into the subject in order to provide prolonged, non-systemic treatment in the subject. Pursuant to such methods, the subcutaneous depot of detomidine or salt thereof may form in the subcutaneous tissue (hypodermis), the epidermis, or the dermis of a subject's skin. The depot can release a sufficient quantity of the detomidine or salt thereof to the patient such that administration of the formulation to the subject on a once- or twice-daily basis is sufficient to provide the prolonged, non-systemic treatment in the subject. In certain embodiments, the depot forms in a least the epidermal and dermal layers of the subject's skin. In further embodiments, a higher concentration of detomidine is observed in the epidermal layer, as compared to the dermal layer.

In accordance with the presently disclosed methods, the topical administration may be performed using conventional techniques. For example, the administration may be performed by delivering an aliquot of the formulation to a physician's or subject's hand (preferably gloved), which is used to smear and then rub the formulation onto an area of skin on the body part for which treatment is desired. The formulation may be topically administered in the chosen manner on a once-daily or twice-daily basis. When the method comprises applying the formulation on a twice-daily basis, appropriate temporal spacing between applications should be used. For example, if the subject is awake for a 16 hour period of the day, then a first application can be performed in the morning, and a second application can be performed in the evening, for example, prior to retiring to bed.

This invention will be better understood by reference to the experimental details which follow, but those skilled in the art will readily appreciate that the specific experiments detailed are only illustrative of the invention as described more fully in the claims which follow thereafter.

### Aspects

Aspect 1. A topical formulation comprising about 0.001 to about 3 wt % detomidine or a salt thereof and, a carrier that is suitable for topical administration to a human subject's skin, wherein the carrier optionally comprises a water-miscible solubilizer that is present in an amount of 0.1 to 40% by weight of the formulation, wherein the formulation has a pH of 4.5 to 9, wherein said detomidine or a salt thereof, is substantially free of 3-((1H-Imidazol-4-yl)methyl)-2-methylbenzaldehyde.

Aspect 2. The topical formulation according to aspect 1, wherein topical administration of the formulation to a human subject is effective to induce formation of a depot of detomidine or a salt thereof in the human subject's skin.

Aspect 3. The topical formulation according to any preceding aspect comprising 0.008 to 3 wt % of said detomidine or salt thereof.

Aspect 4. The topical formulation according to any preceding aspect comprising 0.01 to 2 wt % of said detomidine or salt thereof.

Aspect 5. The topical formulation according to any preceding aspect comprising 0.01 to 1.5 wt % of said detomidine or salt thereof.

Aspect 6. The topical formulation according to any preceding aspect comprising 0.033 to 1 wt % of said detomidine or salt thereof.

Aspect 7. The topical formulation according to any preceding aspect comprising 0.033 to 0.33 wt % of said detomidine or salt thereof.

Aspect 8. The topical formulation according to any preceding aspect wherein said solubilizer comprises an alcohol.

Aspect 9. The topical formulation according to any preceding aspect wherein said solubilizer comprises a sugar alcohol.

Aspect 10. The topical formulation according to any preceding aspect wherein said solubilizer comprises a diol or a polyol.

Aspect 11. The topical formulation according to any preceding aspect wherein said solubilizer comprises a polyether alcohol.

Aspect 12. The topical formulation according to any preceding aspect wherein said solublizer comprises a fatty acid, an organic solvent, a wax, or an oil.

Aspect 13. The topical formulation according to any preceding aspect wherein said solubilizer comprises glycerol, propylene glycol, or a combination thereof.

Aspect 14. The topical formulation according to any preceding aspect wherein said solubilizer is present in an amount of 0.1 to 40% by weight.

Aspect 15. The topical formulation according to any preceding aspect wherein said solubilizer is present in an amount of 0.1 to 10% by weight.

Aspect 16. The topical formulation according to any preceding aspect wherein the carrier comprises a buffer that is effective to maintain the formulation at a pH of about 5.2 to about 8.5.

Aspect 17. The topical formulation according to any preceding aspect wherein the carrier comprises a buffer that is effective to maintain the formulation at pH of about 5.5 to about 8.2.

Aspect 18. The topical formulation according to any preceding aspect wherein the depot of detomidine or a salt thereof is formed in one or more of the subcutaneous layer, the epidermis, or the dermis of the human subject's skin.

Aspect 19. The topical formulation according to any preceding aspect wherein the depot comprises crystals of the detomidine or salt thereof.

Aspect 20. The topical formulation according to any preceding aspect further comprising another therapeutic agent in addition to the detomidine or salt thereof.

Aspect 21. The topical formulation according to aspect 20 wherein said other therapeutic agent comprises an analgesic.

Aspect 22. The topical formulation according to any preceding aspect further comprising one or more of a thickening agent, a preservative, a permeation enhancer, a wax, an emulsifying agent, an emollient, a humectant, a conditioning agent, antioxidant, and a viscosity regulator.

Aspect 23. The topical formulation according to any preceding aspect wherein the pharmaceutically acceptable salt of detomidine is detomidine HCl.

Aspect 24. The topical formulation according to aspect 23, wherein the detomidine HCl is the monohydrate form.

Aspect 25. The topical formulation according to aspect 23, wherein the detomidine HCl is the anhydrous form.

Aspect 26. The topical formulation according to any preceding aspect wherein the total amount of impurities is not more than 0.1% area, not more than 0.06% area, or not more than 0.02% area, based on HPLC.

Aspect 27. The topical formulation according to any preceding aspect wherein the total amount of impurities is not more than 0.06% area, based on HPLC.

Aspect 28. The topical formulation according to any preceding aspect wherein the detomidine or a pharmaceutically acceptable salt thereof is further substantially free of iso-impurity A (((RS)-(3,4-dimethylphenyl)(1H-imidazol-4-yl)methanol)).

Aspect 29. The topical formulation according to any preceding aspect wherein the detomidine or a pharmaceutically acceptable salt thereof is further substantially free of impurity A ((RS)-(2,3-dimethylphenyl)(1H-imidazol-4-yl)methanol).

Aspect 30. The topical formulation according to any preceding aspect wherein the detomidine or a pharmaceutically acceptable salt thereof is further substantially free of impurity B ((RS)-(1-benzyl-1H-imidazol-5-yl)(2,3-dimethylphenyl)methanol) and/or impurity C (4-[(2,3-dimethylcyclohexyl)methyl]-1H-imidazole).

Aspect 31. A method for providing prolonged, non-systemic treatment of pain in a human subject in need thereof comprising topically administering to the human subject a formulation according to any one of aspects 1-30.

Aspect 32. The method according to aspect 31, wherein said pain is neuropathic pain.

Aspect 33. The method according to aspect 31, wherein said pain is diabetic neuropathic pain.

Aspect 34. The method according to aspect 31, wherein said pain is postherpetic neuralgia.

Aspect 35. A method for providing prolonged, non-systemic treatment of pruritus in a human subject in need thereof comprising topically administering to the human subject a formulation according to any one of aspects 1-30.

Aspect 36. The method according to aspect 35, wherein said pruritus is acute pruritus.

Aspect 37. The method according to aspect 35, wherein said pruritus is chronic pruritus.

Aspect 38. The method according to any of aspects 31-37, wherein the formulation is topically administered to the human subject once- or twice-daily basis.

Aspect 39. A method for forming a subcutaneous depot of detomidine or a salt thereof in a human subject comprising topically administering to the human subject a formulation according to any one of aspects 1-30, wherein the detomidine or salt thereof is released from said depot into said human subject in order to provide prolonged, non-systemic treatment in the human subject.

Aspect 40. The method according to aspect 39 wherein the depot releases a sufficient quantity of the detomidine or salt thereof to the patient such that administration of the formulation to the human subject on a once- or twice-daily basis is sufficient to provide the prolonged, non-systemic treatment in the human subject.

Aspect 41. The formulation according to any one of aspects 1 to 30, comprising up to 1 wt % of said detomidine or salt thereof, producing a blood plasma concentration of no more than about 500 pg/mL of said detomidine or salt thereof in said human subject during the first 48 hours of twice-daily topical administration of said formulation.

Aspect 42. The formulation according to any one of aspects 1 to 30, comprising up to 1 wt % of said detomidine or salt thereof, producing a blood plasma concentration of no more than about 500 pg/mL of said detomidine or salt thereof in said human subject during the first 72 hours of twice-daily topical administration of said formulation.

Aspect 43. The formulation according to any one of aspects 1 to 30, comprising up to 1 wt % of said detomidine or salt thereof, producing a blood plasma concentration of no more than about 500 pg/mL of said detomidine or salt thereof in said human subject during the first 96 hours following topical application of said formulation on said human subject.

Aspect 44. The formulation according to any one of aspects 1 to 30, comprising up to 1 wt % of said detomidine or salt thereof, producing a blood plasma concentration of no more than about 800 pg/mL of said detomidine or salt thereof in said human subject following the first 96 hours of twice-daily topical administration of said formulation.

Aspect 45. The formulation according to any one of aspects 41-44 that generates at least about 120 ng/mg of said detomidine or salt thereof of per cm2 of said human subject's dermis at any point during the first 24 hours of twice-daily topical administration of said formulation.

Aspect 46. The formulation according to any one of aspects 41-45 that generates at least about 180 ng/mg of said detomidine or salt thereof of per cm2 of said human subject's dermis at any point during the first 96 hours of twice-daily topical administration of said formulation.

Aspect 47. The formulation according to any one of aspects 41-46 that generates at least about 1200 ng/mg of said detomidine or salt thereof of per cm2 of said human subject's epidermis at any point during the first 24 hours of twice-daily topical administration of said formulation.

Aspect 48. The formulation according to any one of aspects 41-47 that generates at least about 4800 ng/mg of said detomidine or salt thereof of per cm2 of said human subject's epidermis at any point during the first 24 hours of twice-daily topical administration of said formulation.

Aspect 49. The formulation according to any one of aspects 41-48 that generates at least about 2000 ng/mg of said detomidine or salt thereof of per cm2 of said human subject's epidermis at any point during the first 96 hours and following the first 24 hours of twice-daily topical administration of said formulation.

Aspect 50. The formulation according to any one of aspects 41-49 that generates at least about 2400 ng/mg of said detomidine or salt thereof of per cm2 of said human subject's epidermis at any point during the first 96 hours and following the first 24 hours of twice-daily topical administration of said formulation.

Aspect 51. The formulation according to any one of aspects 1 to 30 or 41 to 49 wherein administering said formulation once- or twice-daily topical to a human subject for up to four days results in a blood plasma concentration in said human subject that is less than that required to achieve a systemic therapeutic effect of the detomidine or salt thereof.

Aspect 52. A topical formulation comprising about 0.001 to about 3 wt % detomidine or a salt thereof and, a carrier suitable for topical application to the skin of a human subject, wherein the formulation is systemically therapeutically effective in the human subject while producing a maximum blood plasma concentration of no more than about 1600 pg/mL after four days of twice daily administration of said formulation and wherein said detomidine or a salt thereof, is substantially free of 3-((1H-Imidazol-4-yl)methyl)-2-methylbenzaldehyde.

Aspect 53. The topical formulation according to aspect 52 wherein said formulation produces a maximum blood plasma concentration of no more than about 1400 pg/mL after three days of twice daily administration of said formulation.

Aspect 54. The topical formulation according to aspect 52 wherein said formulation produces a maximum blood plasma concentration of no more than about 1200 pg/mL after two days of twice daily administration of said formulation.

Aspect 55. The topical formulation according to aspect 52 wherein said formulation produces a maximum blood plasma concentration of no more than about 800 pg/mL after the first day of twice daily administration of said formulation.

Aspect 56. The topical formulation according to aspect 52, wherein administering said formulation once- or twice-daily topical to a human subject for up to four days results in a blood plasma concentration in said human subject that is less than that required to achieve a systemic therapeutic effect of the detomidine or salt thereof.

Aspect 57. The topical formulation according to aspect 52, comprising 0.008 to 3 wt % of said detomidine or salt thereof.

Aspect 58. The topical formulation according to aspect 52, comprising 0.01 to 2 wt % of said detomidine or salt thereof.

Aspect 59. The topical formulation according to aspect 52, comprising 0.01 to 1.5 wt % of said detomidine or salt thereof.

Aspect 60. The topical formulation according to aspect 52, comprising 0.033 to 1 wt % of said detomidine or salt thereof.

Aspect 61. The topical formulation according to aspect 52, comprising 0.033 to 0.33 wt % of said detomidine or salt thereof.

Aspect 62. The topical formulation according to any one of aspects 52 to 61, wherein said carrier comprises a water-miscible solubilizer.

Aspect 63. The topical formulation according to aspect 62, wherein said solubilizer comprises an alcohol.

Aspect 64. The topical formulation according to aspect 62, wherein said solubilizer comprises a sugar alcohol.

Aspect 65. The topical formulation according to aspect 62, wherein said solubilizer comprises a diol or a polyol.

Aspect 66. The topical formulation according to aspect 62, wherein said solubilizer comprises a polyether alcohol.

Aspect 67. The topical formulation according to aspect 62, wherein said solublizer comprises a fatty acid, an organic solvent, a wax, or an oil.

Aspect 68. The topical formulation according to aspect 62, wherein said solubilizer comprises glycerol, propylene glycol, or a combination thereof.

Aspect 69. The topical formulation according to any one of aspects 62 to 68, wherein said solubilizer is present in an amount of 0.1 to 40% by weight.

Aspect 70. The topical formulation according to aspect 69, wherein said solubilizer is present in an amount of 0.1 to 10% by weight.

Aspect 71. The topical formulation according to any one of aspects 52 to 70, wherein said carrier comprises a buffer that is effective to maintain the formulation at pH 4.5 to 9.

Aspect 72. The topical formulation according to aspect 71, wherein the buffer is effective to maintain the formulation at a pH of about 5.2 to about 8.5.

Aspect 73. The topical formulation according to aspect 71, wherein the buffer is effective to maintain the formulation at pH of about 5.5 to about 8.2.

Aspect 74. The topical formulation according to aspect 52, wherein said carrier comprises a buffer and a solubilizer, and wherein the solubilizer and buffer combine to dissolve the detomidine or salt thereof in said formulation at greater than 80%, greater than 85%, greater than 90%, or greater than 95% of the saturation point for the detomidine or salt thereof.

Aspect 75. The topical formulation according to any one of aspects 52 to 74, further comprising a further therapeutic agent in addition to the detomidine or salt thereof.

Aspect 76. The topical formulation according to aspect 75, wherein said further therapeutic agent comprises an analgesic.

Aspect 77. The topical formulation according to any one of aspects 52 to 76, further comprising one or more of a thickening agent, a preservative, a permeation enhancer, a wax, an emulsifying agent, an emollient, a humectant, a conditioning agent, and a viscosity regulator.

Aspect 78. A method for providing prolonged, non-systemic treatment for pain in a human subject in need thereof comprising topically administering to the human subject a formulation according to any one of aspects 41-77.

Aspect 79. The method according to aspect 78, wherein said pain is neuropathic pain.

Aspect 80. The method according to aspect 78, wherein said pain is diabetic neuropathic pain.

Aspect 81. The method according to aspect 78, wherein pain is post-herpetic neuropathy.

Aspect 82. A method for providing prolonged, non-systemic treatment for pruritus in a human subject in need thereof comprising topically administering to the human subject a formulation according to any one of aspects 41-77.

Aspect 83. The method according to aspect 82, wherein said pruritus is acute pruritus.

Aspect 84. The method according to aspect 82, wherein said pruritus is chronic pruritus.

Aspect 85. The method according to aspects 78-84, wherein the formulation is topically administered to the human subject one a once- or twice-daily basis.

Aspect 86. A topical formulation comprising about 0.001 to about 3 wt % detomidine or a salt thereof and, a carrier that is suitable for topical administration to a human subject's skin, wherein the carrier optionally comprises a water-miscible solubilizer that is present in an amount of 0.1 to 40% by weight of the formulation, wherein the formulation has a pH of 5.0 to 8.5 and wherein said topical formulation does comprises less than 4% of the total detomidine content as crystalline detomidine base.

Aspect 87. A topical formulation comprising about 0.001 to about 3 wt % detomidine or a salt thereof and, a carrier that is suitable for topical administration to a human subject's skin, wherein the carrier optionally comprises a water-miscible solubilizer that is present in an amount of 0.1 to 40% by weight of the formulation, wherein the formulation has a pH of 4.5 to 9, wherein said detomidine or a salt thereof, is substantially free of the compound that has a mass-to-charge ratio of 201 and elutes at RRT 0.38 when subjected to high performance liquid chromatography (HPLC), wherein the HPLC method comprises a SunFire C8 column, 100 A, 3.5 μm, 4.6×150 mm, an initial mobile phase that is 70% Ammonium Phosphate buffer solution, pH 7.9, and 30% Acetonitrile, a flow rate of 1.0 mL/min, and UV detection at 220 nm.

Aspect 88. The topical formulation according to aspect 87, wherein topical administration of the formulation to a human subject is effective to induce formation of a depot of detomidine or a salt thereof in the human subject's skin.

Aspect 89. The topical formulation according to any of aspects 87 to 88 comprising 0.008 to 3 wt % of said detomidine or salt thereof.

Aspect 90. The topical formulation according to any of aspects 87 to 89 comprising 0.01 to 2 wt % of said detomidine or salt thereof.

Aspect 91. The topical formulation according to any of aspects 87 to 90 comprising 0.01 to 1.5 wt % of said detomidine or salt thereof.

Aspect 92. The topical formulation according to any of aspects 87 to 91 comprising 0.033 to 1 wt % of said detomidine or salt thereof.

Aspect 93. The topical formulation according to any of aspects 87 to 92 comprising 0.033 to 0.33 wt % of said detomidine or salt thereof.

Aspect 94. The topical formulation according to any of aspects 87 to 93 wherein said solubilizer comprises an alcohol.

Aspect 95. The topical formulation according to any of aspects 87 to 94 wherein said solubilizer comprises a sugar alcohol.

Aspect 96. The topical formulation according any of aspects 87 to 95 wherein said solubilizer comprises a diol or a polyol.

Aspect 97. The topical formulation according any of aspects 87 to 96 wherein said solubilizer comprises a polyether alcohol.

Aspect 98. The topical formulation according to any of aspects 87 to 97 wherein said solubilizer comprises a fatty acid, an organic solvent, a wax, or an oil.

Aspect 99. The topical formulation according to any of aspects 87 to 98 wherein said solubilizer comprises glycerol, propylene glycol, or a combination thereof.

Aspect 100. The topical formulation according any of aspects 87 to 99 wherein said solubilizer is present in an amount of 0.1 to 40% by weight.

Aspect 101. The topical formulation according any of aspects 87 to 100 wherein said solubilizer is present in an amount of 0.1 to 10% by weight.

Aspect 102. The topical formulation according any of aspects 87 to 101 wherein the carrier comprises a buffer that is effective to maintain the formulation at a pH of about 5.2 to about 8.5.

Aspect 103. The topical formulation according to any of aspects 87 to 102 wherein the carrier comprises a buffer that is effective to maintain the formulation at pH of about 5.5 to about 8.2.

Aspect 104. The topical formulation according to any of aspects 87 to 103 wherein the depot of detomidine or a salt thereof is formed in one or more of the subcutaneous layer, the epidermis, or the dermis of the human subject's skin.

Aspect 105. The topical formulation according any of aspects 87 to 104 wherein the depot comprises crystals of the detomidine or salt thereof.

Aspect 106. The topical formulation according any of aspects 87 to 105 further comprising another therapeutic agent in addition to the detomidine or salt thereof.

Aspect 107. The topical formulation according to aspect 106 wherein said other therapeutic agent comprises an analgesic.

Aspect 108. The topical formulation according to any of aspects 87 to 107 further comprising one or more of a thickening agent, a preservative, a permeation enhancer, a wax, an emulsifying agent, an emollient, a humectant, a conditioning agent, antioxidant, and a viscosity regulator.

Aspect 109. The topical formulation according to any of aspects 87 to 108 wherein the pharmaceutically acceptable salt of detomidine is detomidine HCl.

Aspect 110. The topical formulation according to aspect 109, wherein the detomidine HCl is the monohydrate form.

Aspect 111. The topical formulation according to aspect 109, wherein the detomidine HCl is the anhydrous form.

Aspect 112. The topical formulation according to any of aspects 87 to 111 wherein the total amount of impurities is not more than 0.1% area, not more than 0.06% area, or not more than 0.02% area, based on HPLC.

Aspect 113. The topical formulation according to any of aspects 87 to 112 wherein the total amount of impurities is not more than 0.06% area, based on HPLC.

Aspect 114. The topical formulation according to any of aspects 87 to 113 wherein the detomidine or a pharmaceutically acceptable salt thereof is further substantially free of iso-impurity A (((RS)-(3,4-dimethylphenyl)(1H-imidazol-4-yl)methanol)).

Aspect 115. The topical formulation according to any of aspects 87 to 114 wherein the detomidine or a pharmaceutically acceptable salt thereof is further substantially free of impurity A ((RS)-(2,3-dimethylphenyl)(1H-imidazol-4-yl)methanol).

Aspect 116. The topical formulation according to any of aspects 87 to 115 wherein the detomidine or a pharmaceutically acceptable salt thereof is further substantially free of impurity B ((RS)-(1-benzyl-1H-imidazol-5-yl)(2,3-dimethylphenyl)methanol) and/or impurity C (4-[(2,3-dimethylcyclohexyl)methyl]-1H-imidazole).

Aspect 117. A method for providing prolonged, non-systemic treatment of pain in a human subject in need thereof comprising topically administering to the human subject a formulation according to any of aspects 87 to 116.

Aspect 118. The method according to aspect 117, wherein said pain is neuropathic pain.

Aspect 119. The method according to aspect 117, wherein said pain is diabetic neuropathic pain.

Aspect 120. The method according to aspect 117, wherein said pain is postherpetic neuralgia.

Aspect 121. A method for providing prolonged, non-systemic treatment of pruritus in a human subject in need thereof comprising topically administering to the human subject a formulation according to any of aspects 87 to 116.

Aspect 122. The method according to aspect 121, wherein said pruritus is acute pruritus.

Aspect 123. The method according to aspect 122, wherein said pruritus is chronic pruritus.

Aspect 124. The method according to any of aspects 117 to 123, wherein the formulation is topically administered to the human subject once- or twice-daily basis.

Aspect 125. A method for forming a subcutaneous depot of detomidine or a salt thereof in a human subject comprising topically administering to the human subject a formulation according to any of aspects 87 to 116, wherein the detomidine or salt thereof is released from said depot into said human subject in order to provide prolonged, non-systemic treatment in the human subject.

Aspect 126. The method according to aspect 125 wherein the depot releases a sufficient quantity of the detomidine or salt thereof to the patient such that administration of the formulation to the human subject on a once- or twice-daily basis is sufficient to provide the prolonged, non-systemic treatment in the human subject.

Aspect 127. The formulation according to any of aspects 87 to 116, comprising up to 1 wt % of said detomidine or salt thereof, producing a blood plasma concentration of no more than about 500 pg/mL of said detomidine or salt thereof in said human subject during the first 48 hours of twice-daily topical administration of said formulation.

Aspect 128. The formulation according to any of aspects 87 to 116, comprising up to 1 wt % of said detomidine or salt thereof, producing a blood plasma concentration of no more than about 500 pg/mL of said detomidine or salt thereof in said human subject during the first 72 hours of twice-daily topical administration of said formulation.

Aspect 129. The formulation according to any of aspects 87 to 116, comprising up to 1 wt % of said detomidine or salt thereof, producing a blood plasma concentration of no more than about 500 pg/mL of said detomidine or salt thereof in said human subject during the first 96 hours following topical application of said formulation on said human subject.

Aspect 130. The formulation according to any of aspects 87 to 116, comprising up to 1 wt % of said detomidine or salt thereof, producing a blood plasma concentration of no more than about 800 pg/mL of said detomidine or salt thereof in said human subject following the first 96 hours of twice-daily topical administration of said formulation.

Aspect 131. The formulation according to any of aspects 127 to 130 that generates at least about 120 ng/mg of said detomidine or salt thereof of per cm2 of said human subject's dermis at any point during the first 24 hours of twice-daily topical administration of said formulation.

Aspect 132. The formulation according to any one of aspects 127 to 131 that generates at least about 180 ng/mg of said detomidine or salt thereof of per cm2 of said human subject's dermis at any point during the first 96 hours of twice-daily topical administration of said formulation.

Aspect 133. The formulation according to any one of aspects 127 to 132 that generates at least about 1200 ng/mg of said detomidine or salt thereof of per cm2 of said human subject's epidermis at any point during the first 24 hours of twice-daily topical administration of said formulation.

Aspect 134. The formulation according to any one of aspects 127 to 133 that generates at least about 4800 ng/mg of said detomidine or salt thereof of per cm2 of said human subject's epidermis at any point during the first 24 hours of twice-daily topical administration of said formulation.

Aspect 135. The formulation according to any one of aspects 127 to 134 that generates at least about 2000 ng/mg of said detomidine or salt thereof of per cm2 of said human subject's epidermis at any point during the first 96 hours and following the first 24 hours of twice-daily topical administration of said formulation.

Aspect 136. The formulation according to any one of aspects 127 to 135 that generates at least about 2400 ng/mg of said detomidine or salt thereof of per cm2 of said human subject's epidermis at any point during the first 96 hours and following the first 24 hours of twice-daily topical administration of said formulation.

Aspect 137. The formulation according to any one of aspects 87 to 116 or 127 to 135 wherein administering said formulation once- or twice-daily topical to a human subject for up to four days results in a blood plasma concentration in said human subject that is less than that required to achieve a systemic therapeutic effect of the detomidine or salt thereof.

Aspect 138. A topical formulation comprising about 0.001 to about 3 wt % detomidine or a salt thereof and, a carrier suitable for topical application to the skin of a human subject, wherein the formulation is systemically therapeutically effective in the human subject while producing a maximum blood plasma concentration of no more than about 1600 pg/mL after four days of twice daily administration of said formulation and wherein said detomidine or a salt thereof, is substantially free of the compound that has a mass-to-charge ratio of 201 and elutes at RRT 0.38 when subjected to high performance liquid chromatography (HPLC), wherein the HPLC method comprises a SunFire C8 column, 100Å, 3.5 μm, 4.6×150 mm, an initial mobile phase that is 70% Ammonium Phosphate buffer solution, pH 7.9, and 30% Acetonitrile, a flow rate of 1.0 mL/min, and UV detection at 220 nm.

Aspect 139. The topical formulation according to aspect 138 wherein said formulation produces a maximum blood plasma concentration of no more than about 1400 pg/mL after three days of twice daily administration of said formulation.

Aspect 140. The topical formulation according to aspect 138 wherein said formulation produces a maximum blood plasma concentration of no more than about 1200 pg/mL after two days of twice daily administration of said formulation.

Aspect 141. The topical formulation according to aspect 138 wherein said formulation produces a maximum blood plasma concentration of no more than about 800 pg/mL after the first day of twice daily administration of said formulation.

Aspect 142. The topical formulation according to aspect 138, wherein administering said formulation once- or twice-daily topical to a human subject for up to four days results in a blood plasma concentration in said human subject that is less than that required to achieve a systemic therapeutic effect of the detomidine or salt thereof.

Aspect 143. The topical formulation according to aspect 138, comprising 0.008 to 3 wt % of said detomidine or salt thereof.

Aspect 144. The topical formulation according to aspect 138, comprising 0.01 to 2 wt % of said detomidine or salt thereof.

Aspect 145. The topical formulation according to aspect 138, comprising 0.01 to 1.5 wt % of said detomidine or salt thereof.

Aspect 146. The topical formulation according to aspect 138, comprising 0.033 to 1 wt % of said detomidine or salt thereof.

Aspect 147. The topical formulation according to aspect 138, comprising 0.033 to 0.33 wt % of said detomidine or salt thereof.

Aspect 148. The topical formulation according to any one of aspects 138 to 147, wherein said carrier comprises a water-miscible solubilizer.

Aspect 149. The topical formulation according to aspect 148, wherein said solubilizer comprises an alcohol.

Aspect 150. The topical formulation according to aspect 148, wherein said solubilizer comprises a sugar alcohol.

Aspect 151. The topical formulation according to aspect 148, wherein said solubilizer comprises a diol or a polyol.

Aspect 152. The topical formulation according to aspect 148, wherein said solubilizer comprises a polyether alcohol.

Aspect 153. The topical formulation according to aspect 148, wherein said solublizer comprises a fatty acid, an organic solvent, a wax, or an oil.

Aspect 154. The topical formulation according to aspect 148, wherein said solubilizer comprises glycerol, propylene glycol, or a combination thereof.

Aspect 155. The topical formulation according to any one of aspects 148 to 154, wherein said solubilizer is present in an amount of 0.1 to 40% by weight.

Aspect 156. The topical formulation according to aspect 155, wherein said solubilizer is present in an amount of 0.1 to 10% by weight.

Aspect 157. The topical formulation according to any one of aspects 148 to 156, wherein said carrier comprises a buffer that is effective to maintain the formulation at pH 4.5 to 9.

Aspect 158. The topical formulation according to aspect 157, wherein the buffer is effective to maintain the formulation at a pH of about 5.2 to about 8.5.

Aspect 159. The topical formulation according to aspect 157, wherein the buffer is effective to maintain the formulation at pH of about 5.5 to about 8.2.

Aspect 160. The topical formulation according to aspect 148, wherein said carrier comprises a buffer and a solubilizer, and wherein the solubilizer and buffer combine to dissolve the detomidine or salt thereof in said formulation at greater than 80%, greater than 85%, greater than 90%, or greater than 95% of the saturation point for the detomidine or salt thereof.

Aspect 161. The topical formulation according to any one of aspects 148 to 160, further comprising a further therapeutic agent in addition to the detomidine or salt thereof.

Aspect 162. The topical formulation according to aspect 161, wherein said further therapeutic agent comprises an analgesic.

Aspect 163. The topical formulation according to any one of aspects 148 to 162, further comprising one or more of a thickening agent, a preservative, a permeation enhancer, a wax, an emulsifying agent, an emollient, a humectant, a conditioning agent, and a viscosity regulator.

Aspect 164. A method for providing prolonged, non-systemic treatment for pain in a human subject in need thereof comprising topically administering to the human subject a formulation according to any one of aspects 127 to 163.

Aspect 165. The method according to aspect 164, wherein said pain is neuropathic pain.

Aspect 166. The method according to aspect 164, wherein said pain is diabetic neuropathic pain.

Aspect 167. The method according to aspect 164, wherein pain is post-herpetic neuropathy.

Aspect 168. A method for providing prolonged, non-systemic treatment for pruritus in a human subject in need thereof comprising topically administering to the human subject a formulation according to any one of aspects 127 to 163.

Aspect 169. The method according to aspect 168, wherein said pruritus is acute pruritus.

Aspect 170. The method according to aspect 168, wherein said pruritus is chronic pruritus.

Aspect 171. The method according to aspects 164 to 170, wherein the formulation is topically administered to the human subject one a once- or twice-daily basis.

### EXAMPLES

### Example 1: Characterization of Commercially Sourced Material

Anhydrous detomidine HCl was sourced from two commercial API suppliers. Samples of the anhydrous detomidine products were analyzed for water content and characterized by microscope, XRPD and thermal analyses. The results are summarized in Table 1.

TABLE 1
Characterization of commercial anhydrous detomidine HCl

| | | | LOD by |
|---|---|---|---|
| | Form by | m.p. by DSC | TGA |
| Batch | XRPD | ° C. | % |
| Non-GMP, | Mixture^{a} | Two peaks at 95° and 160° | 2.7 |
| Off-white | | | |
| GMP1, white | Mixture^{a} | Two peaks at 95.5° and 157° | 0.7 |
| GMP2, white | Mixture^{a} | A tiny peak at 95° and peak | 0.3 |
| | | a 159.6° | |
| GMP3, white | Mixture^{a} | Two peaks at 95° and 159° | 0.6 |

^{a}Anhydrous + monohydrate

The values presented in Table 1 demonstrate that the commercial samples of detomidine HCl labeled as anhydrous contain some amount of monohydrate and this amount varied depending on storage conditions and packaging.

### Example 2: Stability Assessment of Anhydrate and Monohydrate Forms of Detomidine Base and Detomidine HCl

Pure forms of crystalline free base, and HCl salt (both monohydrate and anhydrate) were prepared from commercially sourced anhydrous detomidine HCl as outlined in Table 1, and characterized using XRPD and thermal analysis. The solids were crystallized from aqueous solutions and then dried under different conditions. The crystallization and drying conditions are summarized in Table 2.

TABLE 2
Preparation of detomidine HCl crystalline forms

| | | Drying conditions | | | |
|---|---|---|---|---|---|
| Sample | | | P; | Time; | Yield |
| No. | Preparation procedure | T ° C. | mbar | hrs | % |
| 1 | Re-crystallization of anhydrous | RT | 20 | 22 | 84.5 |
| | detomidine hydrochloride from | | | | |
| | 2.88 vol. water | | | | |
| 2 | Re-crystallization of anhydrous | 90 | 30-40 | 25 | 84 |
| | detomidine hydrochloride from | | | | |
| | 2.88 vol. water | | | | |
| 3 | Drying under vacuum | 90 | 30-40 | 17 | 100 |
| 4 | Splitting of detomidine | 45 | 30-40 | 13 | 85.2 |
| | hydrochloride with NaOH in | | | | |
| | water, precipitation and | | | | |
| | isolation of detomidine free base. | | | | |
| | Reaction of detomidine free base | | | | |
| | with HCl 1.05 eq. in 2.80 vol. | | | | |
| | water | | | | |
| 5 | Carbon treatment of detomidine | 45 | 30-40 | 15.5 | 84.5 |
| | hydrochloride in water, splitting | | | | |
| | with NaOH, precipitation and | | | | |
| | isolation of detomidine free base. | | | | |
| | Reaction of detomidine free base | | | | |
| | with HCl 1.05 eq. in 2.80 vol. | | | | |
| | water | | | | |

The properties of the solids crystallized according to Table 2 are described in Table 3.

TABLE 3
Properties of Detomidine HCl crystalline forms

| | | m.p. | | LOD | | | Particle |
|---|---|---|---|---|---|---|---|
| Sam- | | open | | by | | Water | habit, by |
| ple | | capillary | | TGA | DSC | by cKF | micro- |
| No. | Color | ° C. | XRPD | % | ° C. | % | scope |
| 1 | Off- | 158.8- | Monohydrate | 7.66 | Two | 7.36 | Large |
| | white | 159.8 | | | melting | | regular |
| | | | | | peaks | | prisms |
| | | | | | 94°, | | |
| | | | | | 140° | | |
| 2 | Off- | 159.8- | Anhydrous | 0.04 | Melting | N.A. | Small |
| | white | 160.6 | | | at 160° | | irregular |
| | | | | | | | particles |
| 3 | Off- | 158.6- | Anhydrous | 0.3 | Melting | N.A. | Small |
| | white | 160.1 | | | at 160° | | irregular |
| | | | | | | | particles |
| 4 | Off- | N.A. | Monohydrate | 7.16 | Two | N.A. | Large |
| | white | | | | melting | | regular |
| | | | | | peaks | | prisms |
| | | | | | 94º, | | and |
| | | | | | 157° | | rods |
| 5 | White | N.A. | Monohydrate | N.A. | N.A. | 7.46 | Large |
| | | | | | | | regular |
| | | | | | | | prisms |
| | | | | | | | and |
| | | | | | | | rods |

These results demonstrate that crystallization from 2.8-2.9 volumes of water is effective for isolation and purification of the detomidine HCl monohydrate drug substance. Drying of the monohydrate under mild conditions (20-40 mbar and temperatures from at least ambient to about 45° C.) provided pure monohydrate without traces of the anhydrous form.

The same monohydrate dried at elevated temperature (30-40 mbar 90° C.) converted completely into the anhydrous form. The vacuum dried, hermetically closed anhydrate did not absorb water from the atmosphere and did not convert into the monohydrate. After exposure to atmospheric air, however, the anhydrate absorbed water and converted to a mixture of anhydrate and monohydrate.

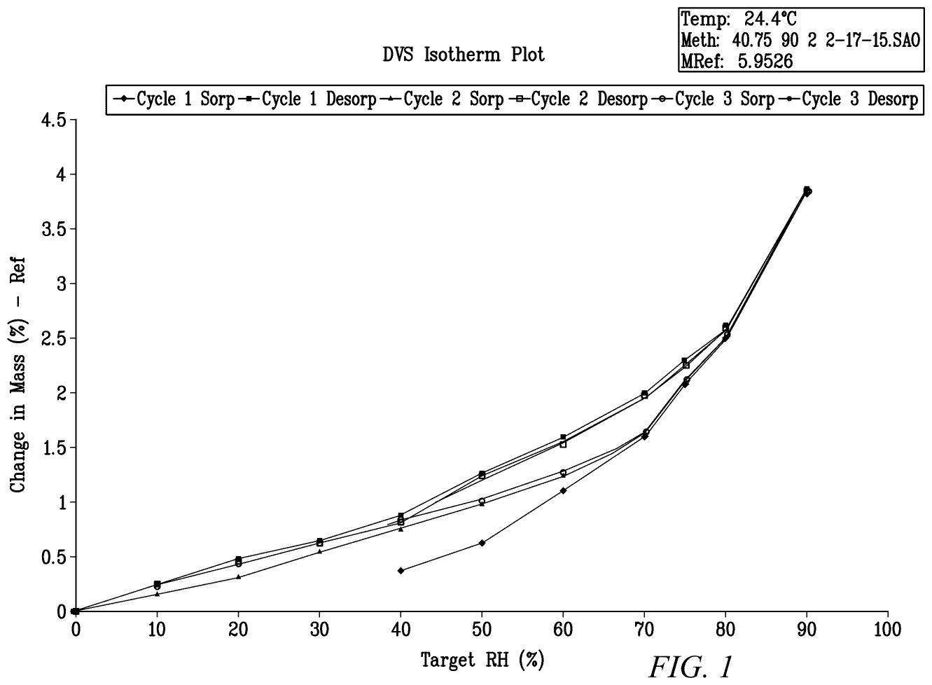

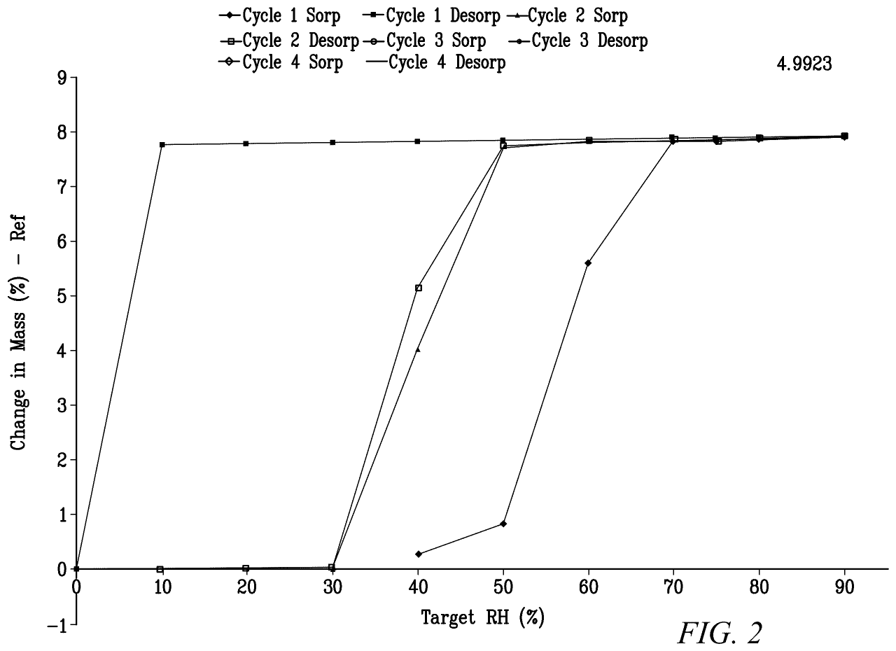

Melting points (m.p.) of the intermediate detomidine free base and hydrochloride of Sample 5 measured in open capillary corresponded with the published literature and the DSC data and are presented in Table 4. In order to evaluate effect of humidity on different forms of detomidine, a hydration study was performed. Samples of detomidine free base and hydrochloride salt were subjected to DVS analysis. These observations are in accordance with the DVS results shown in FIGS. 1 and 2, for detomidine free base and detomidine HCl, respectively.

TABLE 4
Composition and properties of known solid forms of detomidine

| | | | | | m.p., open |
|---|---|---|---|---|---|
| | | Content, % wt. | | | capillary |
| Solid form | MW | Base | HCl | Water | ° C. |
| Free base | 186.2 | 100 | 0 | 0 | 118-119 |
| | | | | | (114-116)^{a} |
| Hydrochloride anhydrous | 222.7 | 83.6 | 16.4 | 0 | 158-160 |
| Hydrochloride | 240.7 | 77.3 | 15.2 | 7.5 | 159-160 |
| monohydrate | | | | | (160-161)^{a} |

^{a}literature data

The free base was found to be crystalline and insoluble in water but it reacted readily with aqueous HCl giving soluble detomidine hydrochloride.

Crystallization from water provided effective purification of the detomidine HCl and formation of large regular crystals. Anhydrous detomidine hydrochloride appeared as small irregular particles whereas the possibility to control particle size distribution by crystallization parameters existed for the monohydrate.

The detomidine free base was found to be non-hygroscopic, but also able to absorb more than 1% of water at relative humidity (RH)>50%. An increase of humidity from RH 70% to RH>90% did not lead to absorption of additional water to monohydrate. During the dehydration cycle, the monohydrate began to lose water at RH˜10% and converted into the anhydrate at RH=0%. Anhydrate did not absorb water at RH<30% and transformed completely to into the monohydrate at RH between 30% and 50%.

Four cycles of hydration-dehydration demonstrated good reproducibility of anhydrate-monohydrate interconversion.

An anhydrous detomidine HCl of Sample 2 was shown to absorb water to a level of cKF 7.7% which corresponds well to the theoretical amount of water in the monohydrate form (Table 4). The hydration profile of detomidine hydrochloride showed that the monohydrate is stable in a wide range of humidity between 10% and >90% RH. At the same time, the anhydrous form is not stable in atmospheric air and absorbs water at RH=30-50%.

This data demonstrates that the anhydrous form is challenging in the aspects of water content and solid form stability and that detomidine HCl monohydrate is more suitable for pharmaceutical development.

### Example 3: Impurity Analysis of Commercially Sourced Detomidine HCl

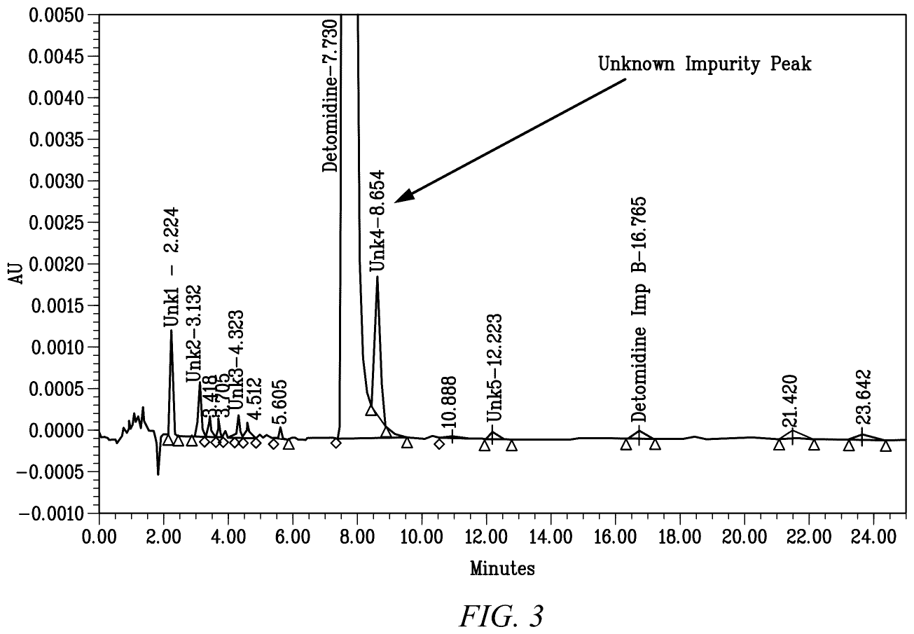

Using the established Pharmacopeia HPLC protocol (Symmetry C8, 5 μm, 4.6×150 mm column, with a mobile phase of 65% Ammonium phosphate buffer pH 7.9 and 35% Acetonitrile at a flow rate of 1.0 mL/min and UV detection at 220 nm), sourced samples of detomidine HCl were assayed for impurities. As shown in FIG. 3, a previously unreported peak was identified, which partially overlapped with that of detomidine. By LC-MS/MS analysis, this impurity was shown to have the same molecular weight as detomidine.

The established Pharmacopeia HPLC protocol did not separate the detomidine from the impurity. Therefore, for further identification of the elusive impurity, new HPLC protocols for assaying detomidine HCl were developed. One protocol (“HPLC Protocol A”) comprised using a SunFire C8 column, 100Å, 3.5 μm, 4.6×150 mm column with an initial mobile phase of 70% Ammonium Phosphate buffer solution, pH 7.9 and 30% Acetonitrile, at a flow rate of 1.0 mL/min and UV detection at 220 nm. To remove late eluting peaks, the flush gradient shown in Table 5 was applied after each run. This HPLC protocol allowed for a resolution factor of 3.9 between detomidine and the unidentified impurity. The quantitation level (QL) for impurities and degradation products is 0.025%. The detection level (DL) for impurities and degradation products is 0.01%.

TABLE 5
Flush gradient for HPLC protocol

| | % Ammonium Phosphate | |
|---|---|---|
| Time, min | buffer solution | % Acetonitrile |
| 0 | 70 | 30 |
| 25 | 70 | 30 |
| 27 | 30 | 70 |
| 32 | 30 | 70 |
| 33 | 70 | 30 |
| 35 | 70 | 30 |

Given its molecular weight, it was hypothesized that the impurity was iso-detomidine.

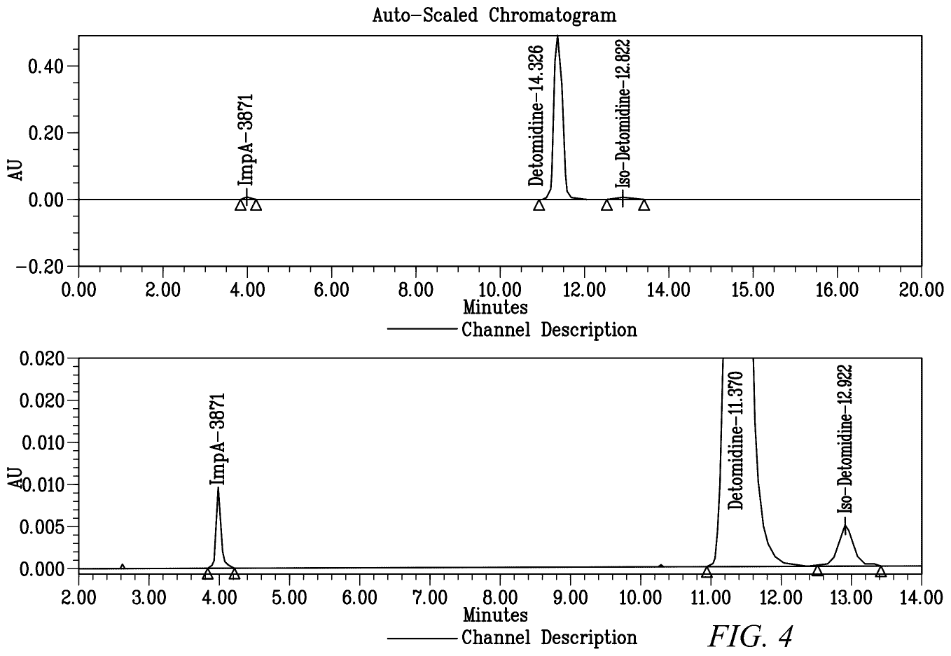

A solution of 100 μg/ml detomidine HCl and about 1 μg/mL (about 2% of the working concentration) of detomidine impurity A and iso-detomidine were prepared and assayed using the new HPLC protocol (HPLC Protocol A), disclosed hereinabove. FIG. 4 is a chromatogram showing that the previously unreported peak is confirmed as being iso-detomidine.

The analysis of commercially sourced detomidine HCl revealed a significant additional impurity. Table 6 provides levels of the various detomidine impurities in different commercial batches. In all batches, total impurities were observed at levels of >0.1% area.

TABLE 6
Impurity levels (% area) in commercial batches of detomidine.

| | | | impurity | | iso- | Total |
|---|---|---|---|---|---|---|
| Batch | | Pro- | A (RRT = | RRT = | deto- | Impu- |
| # | Form | duction | 0.33) | 0.38 | midine | rities |
| i | Anhydrous | Non-GMP | 0.07 | ND | 0.21 | 0.32 |
| ii | Anhydrous | Non-GMP | 0.15 | ND | 0.02 | 0.19 |
| iii | Monohydrate + | GMP | 0.03 | 0.02 | 0.02 | 0.11 |
| | Anhydrous | | | | | |
| | (15:85) | | | | | |
| iv | Monohydrate | GMP | 0.04 | 0.17 | 0.01 | 0.3 |
| V | Monohydrate¹ | GMP | 0.01 | 0.01 | ND | 0.04 |

¹provided by commercial supplier after undergoing the recrystallization process of Example 4, provided by inventors.

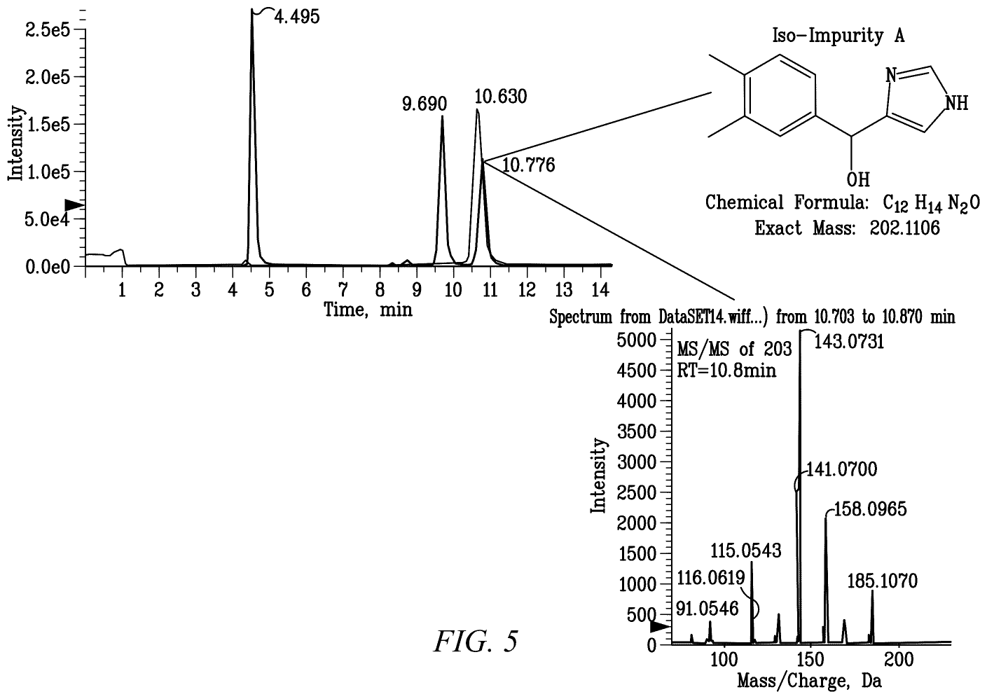

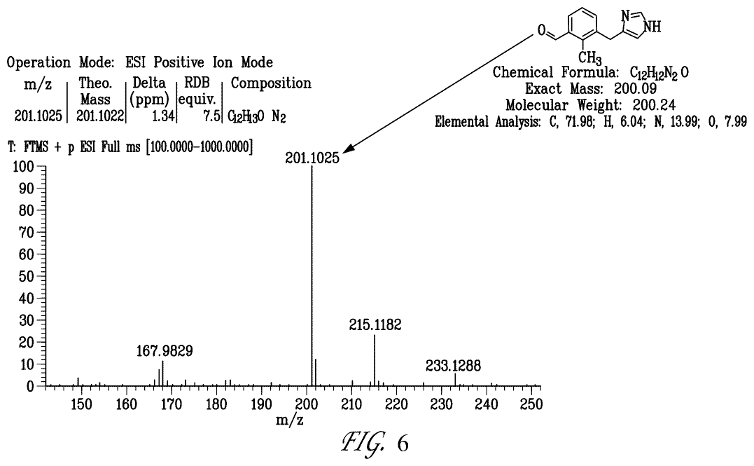

Further analysis of the peak at RRT=0.38 showed that it actually consisted of 2 separate, overlapping peaks. As shown in FIG. 5, LC-MS/MS analysis confirmed one of these peaks as iso-impurity A. After isolation and purification by preparative HPLC, further analysis, using ¹H and ¹³C NMR, LC-MS and HR-ESI-MS and as shown in FIG. 6, identified the second peak as 3-((1H-Imidazol-4-yl)methyl)-2-methylbenzaldehyde.

### Example 4: Purification of Organic Impurities from Detomidine HCl Monohydrate

Two potential procedures for purification of organic impurities from sourced monohydrate were compared. The first attempted procedure was by direct re-crystallization of detomidine HCl from 2.88 volumes of water, while the second included carbon treatment and precipitation of detomidine free base followed by the free base being reacted with HCl and crystallized as monohydrate. Both procedures used the same non-GMP, off white anhydrous detomidine HCl starting material which had previously been shown in Table 6 to contain 0.21% of iso-detomidine and 0.07% of Impurity A. All the re-crystallized materials were found to have practically the same purity level. The direct re-crystallization procedure was found to provide a product with a high yield and purity and at the same time provides a practical and scalable crystallization process which could be controlled by process parameters such as seeding and cooling rate.

### Example 4a: Direct Recrystallization

Anhydrous detomidine HCl (4.5 g), as described in Example 3, Batch #1, was introduced to a round-bottom flask with a magnetic stirrer and thermometer. Deionized water (13 ml) was then added and the mixture stirred and heated in a water bath. At 39° C., the complete dissolution of solids was observed, providing a clear yellow solution with a pH=4.

The batch was gradually cooled by stirring. At 31° C., intensive crystallization was observed. The resulting slurry was cooled in an ice-water bath for 20 min and filtered. Flask and cake were then washed with 2 ml of cold deionized water and 3.97 g of a white to cream colored solid was collected. 2.03 g of the material was dried in a vacuum desiccator at ambient temperature and 20 mbar to a constant weight over 23 hrs producing a dry monohydrate—1.96 g off-white crystalline solid (sample 1).

An additional 1.91 g of the material was dried in a vacuum oven at 90° C. under house vacuum to a constant weight over about 24.5 hrs producing a dry anhydrate, 1.68 g off-white solid (sample 2).

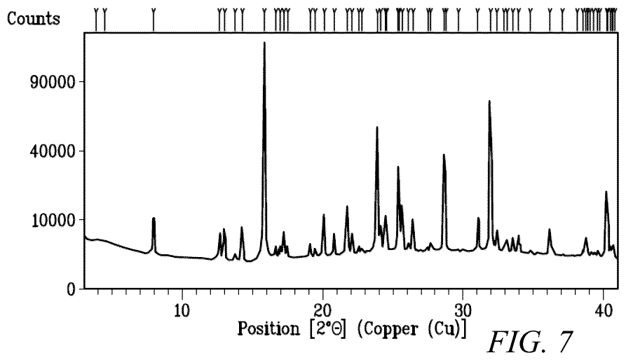

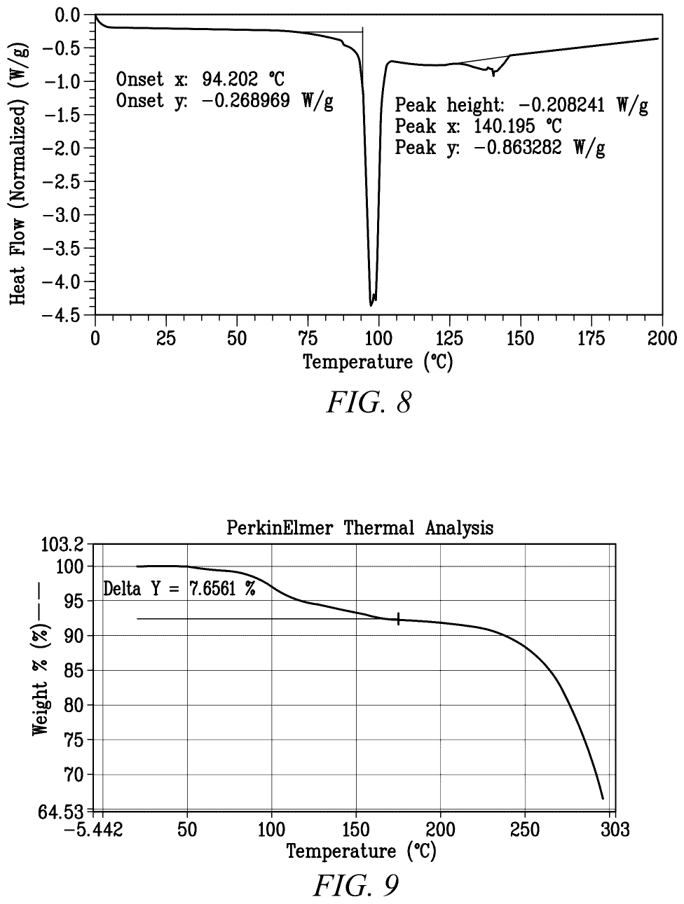

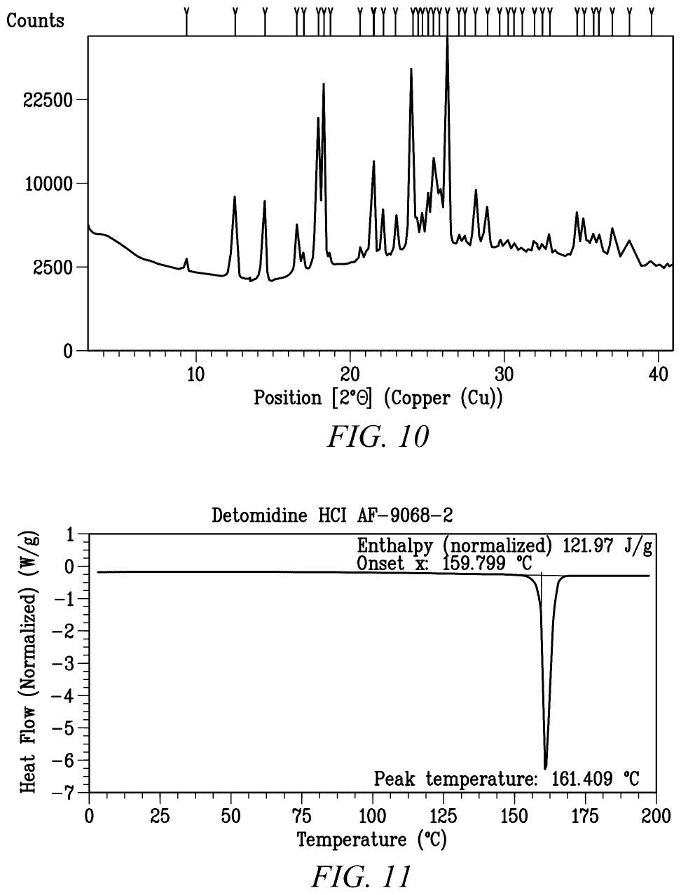

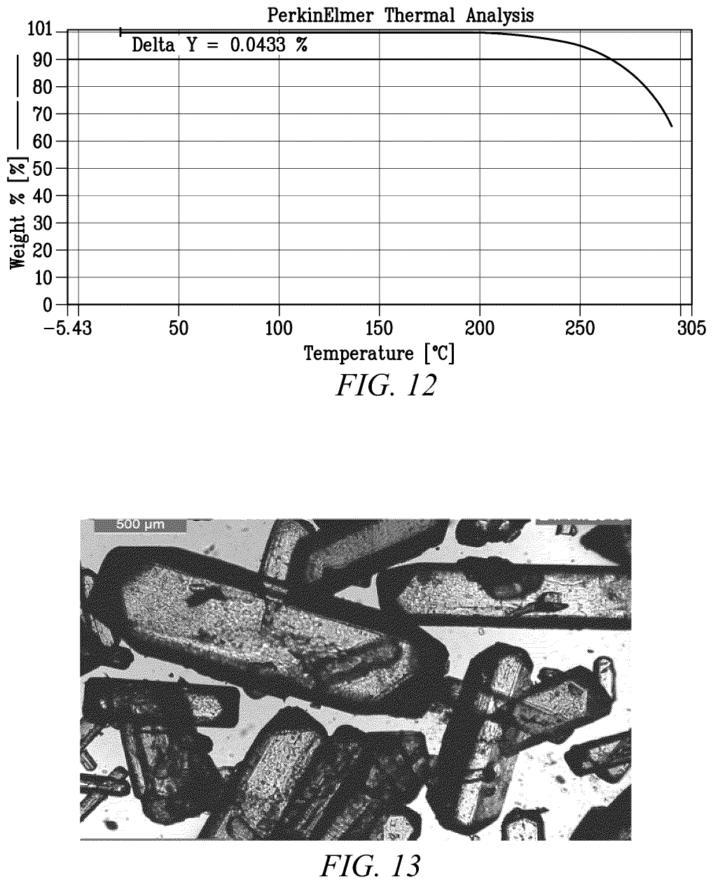

The two samples were subjected to physical characterization and purity analysis by HPLC. The XRPD spectra and DSC and TGA thermograms of sample 1 are presented in FIGS. 7-9 and of sample 2 are presented in FIGS. 10-12, respectively.

As shown in Table 7, direct re-crystallization resulted in the effective purification from all organic impurities, but was not effective for color. The content of iso-detomidine and of Impurity A was reduced to a level below the QL, but the off white color remained after re-crystallization.

TABLE 7
properties following direct recrystallization (sample 1)

| | | |
|---|---|---|
| | Before | After |
| | re-crystallization | re-crystallization |
| Color | Off-white | Off-white |

Impurities by HPLC, % area

| | | |
|---|---|---|
| RRT = 0.20-0.21% area | 0.06 | 0.02 |
| Impurity A (RRT = 0.33) | 0.07 | 0.01¹ |
| RRT = 0.48% area² | 0.01 | ND |
| iso-detomidine (RRT = 1.14) | 0.21 | 0.01¹ |
| Total Impurities | 0.32 | 0.02 |

¹below the QL
²system peak

### Example 4b(i): Carbon Treatment and Detomidine Free Base Isolation

Anhydrous detomidine HCl (70.3 g), as described in Example 3, Batch #1, and deionized water (220 ml) were introduced to a 0.5 liter jacketed glass reactor equipped with a mechanical stirrer, thermocoupler and a circulating oil bath for heating and cooling.

The mixture was heated while stirring. At 40° C., complete dissolution was observed. Active carbon (CXV type, 5.2 g) was added to the clear yellow solution and the batch stirred at 45° C. for 50 minutes. Following this, the batch was filtered on through paper filter on Buchner funnel, reactor and filter washed with deionized water (20 ml).

The slightly yellowish clear filtrate was reintroduced to the 0.5 liter reactor, stirred and 40% NaOH solution was added at 40° C. After 10 ml NaOH solution was added, a pH of 7 was reached and precipitation began. An additional 13 ml of NaOH was added over 1 hour at 42-52° C. and intensive stirring (400-450 rpm) performed. The mixture at the end of the addition of NaOH had a pH of 13.

The batch was stirred at 33-35° C. overnight then cooled to 16° C. over 4 hours and stirred at this temperature for an additional hour. The resultant solid was filtered on Buchner filter, reactor and cake washed with two portions of deionized water (2×200 ml). The wet solid (86 g) was dried in a vacuum oven at 45° C. to constant weight to produce a dry product (53.2 g, Yield 90.7%)—white powder, m.p.=118.6-119.2

The dry detomidine base was analyzed for purity by HPLC, the results presented in Table 8.

TABLE 8
Properties of detomidine base (intermediate in sample 2)

| | | |
|---|---|---|
| | Before | Free base |
| | purification | (dry) |
| Color | Off-white | White |

Impurities by HPLC, % area

| | | |
|---|---|---|
| RRT = 0.20-0.21% area | 0.06 | 0.05 |
| Impurity A (RRT = 0.33) | 0.07 | 0.02 |
| RRT = 0.48% area¹ | 0.01 | 0.01 |
| iso-detomidine (RRT = 1.14% area) | 0.21 | 0.13 |
| Total Impurities | 0.31 | 0.22 |

¹system peak

### Example 4b(ii): Monohydrate Crystallization from Detomidine Base

The dry detomidine free base (53.0 g) from Example 5b(i) was introduced together with 37% HCl (29.7 g) and deionized water (159 g) into a 0.5 liter jacketed glass reactor equipped with a mechanical stirrer, a thermocoupler and a circulating oil bath for heating and cooling. The batch was stirred and heated to 45° C., at 37° C. complete dissolution of solid was observed. The clear solution had a pH of 1. The solution was cooled gradually to 37° C. and seeded with detomidine HCl monohydrate and cooled gradually to 3° C. over 4 hours, and then the batch was stirred for 45 minutes at this temperature.

The solid was filtered on Buchner filter, reactor and cake washed with cold deionized water (80 ml). The wet solid (61.9 g) was dried in vacuum oven for 16 hours at 45° C. to produce a dry product (57.8 g, Yield 84.3%)—white crystalline powder (sample 2)

The dry detomidine HCl monohydrate was analyzed for water by CKF (H₂O=7.46%) and for purity by HPLC with the results presented in Table 9. Microscopic observation for particle morphology (regular prisms) was performed and the microscopic photograph is shown in FIG. 13.

TABLE 9
Properties of detomidine HCl (sample 2)

| | Before | Monohydrate after |
|---|---|---|
| | purification | crystallization |
| Color | Off-white | White |
| Impurities by HPLC, % area | | |
| RRT = 0.20-0.21% area | 0.06 | ND |
| Impurity A (RRT = 0.33% area) | 0.07 | ND |
| RRT = 0.48 % area¹ | 0.01 | ND |
| iso-detomidine (RRT = 1.14% area) | 0.21 | ND |
| Total Impurities | 0.31 | ND |

¹system peak

### Example 4c: Re-Crystallization of Detomidine HCl to Monohydrate, Bench Scale Experiment

Anhydrous detomidine HCl (754.6 g), as described in Example 3, Batch #2, 37% HCl (116.0 g) and deionized water (2008 g) were introduced to a 3 liter glass jacketed reactor equipped with a mechanical stirrer, two baffles, a thermocoupler and a circulating oil bath for heating and cooling. The batch was stirred and heated to 52° C., at 47° C. complete dissolution was observed and the clear solution was found to have a pH of 0-0.5.

The solution was cooled gradually and at 45° C. seeded with detomidine HCl monohydrate (0.5 g). Crystallization initiation was observed at 43° C. and the batch was then cooled to 1.5° C. during 5 hours and stirred for 12 hours at this temperature. The solid was filtered on Buchner filter and conditioned on the filter with vacuum for 40 minutes. The wet product (817 g) was dried in vacuum oven to constant weight.

For the first 13 hours, the material was dried at 30° C. and 35-27 mbar, then for an additional 7 hours at 40° C. and 30-18 mbar to produce a dry product (771.2 g, Yield 94.6%)-white crystalline powder (Batch “90” in Tables 8-9; sample 3)

Dry detomidine HCl monohydrate was analyzed for water by CKF (H₂O=7.37%) and for purity by HPLC, the results presented in Table 10. The physical characterization results are shown in Table 10.

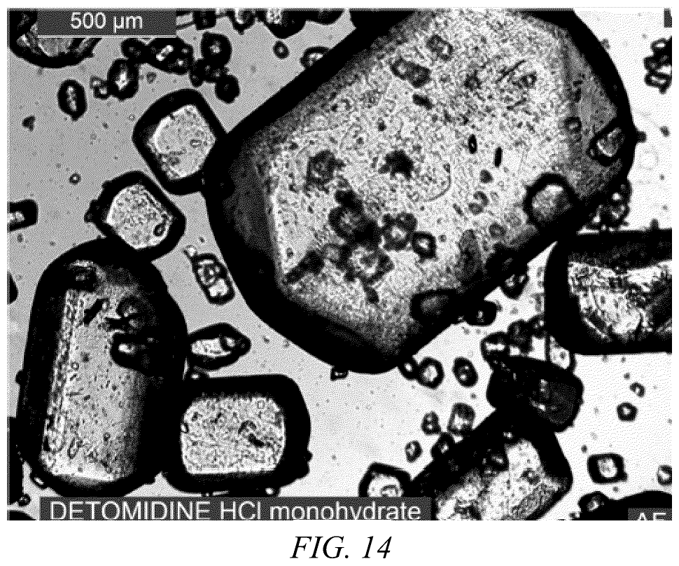

The material was subjected to physical characterization and microscopic observation for particle morphology (regular prisms) microscopic photograph presented in FIG. 14.

TABLE 10
Properties of detomidine HCl (sample 3)

| | | Monohydrate |
|---|---|---|
| | Before | after |
| | re-crystallization | crystallization |
| Color | Non GMP, white | White |
| Impurities by HPLC, % area | | |
| RRT = 0.20-0.21% area | ND | 0.03 |
| Impurity A (RRT = 0.33% area) | 0.15 | 0.03 |
| RRT = 0.481% area | 0.01 | ND |
| RRT = 0.611% area | ND | ND |
| iso-detomidine (RRT = 1.14% area) | 0.02 | ND |
| Total Impurities | 0.19 | 0.06 |

¹system peak

### Example 5: Properties of Solid Form Detomidine Base

### Example 5a: X-Ray Powder Diffraction (XRPD) Kα1Mode

XRPD patterns were recorded on a PANalytical X Pert Pro diffractometer equipped with an Xcelerator detector using Cu Kα radiation at 45 kV and 40 mA.

Kα1 radiation was obtained with a highly oriented crystal (Ge111) incident beam monochromator. A 10 mm beam mask, and divergence (¼°), and anti-scatter (⅛°) slits were inserted on the incident beam side. Receiving (5 mm) and Soller (0.04 rad.) slits were inserted on the diffracted beam side. The XRPD scan was collected from ca. 2 to 40° 20 with a 0.00800 step size and 96.06 sec counting time which resulted in a scan rate of approximately 0.5°/min. The sample was spread on a glass plate or a silicon zero background (ZBG) plate for the measurement. The sample was rotated at 15 revolutions/min on a PANalytical PW3065/12 Spinner. Measurement of the Si reference standard before the data collection resulted in values for 20 and intensity that were well within the tolerances of 28.38<20<28.50 and significantly greater than the minimum peak height of 150 cps.

### Example 5b: XRPD Standard Mode

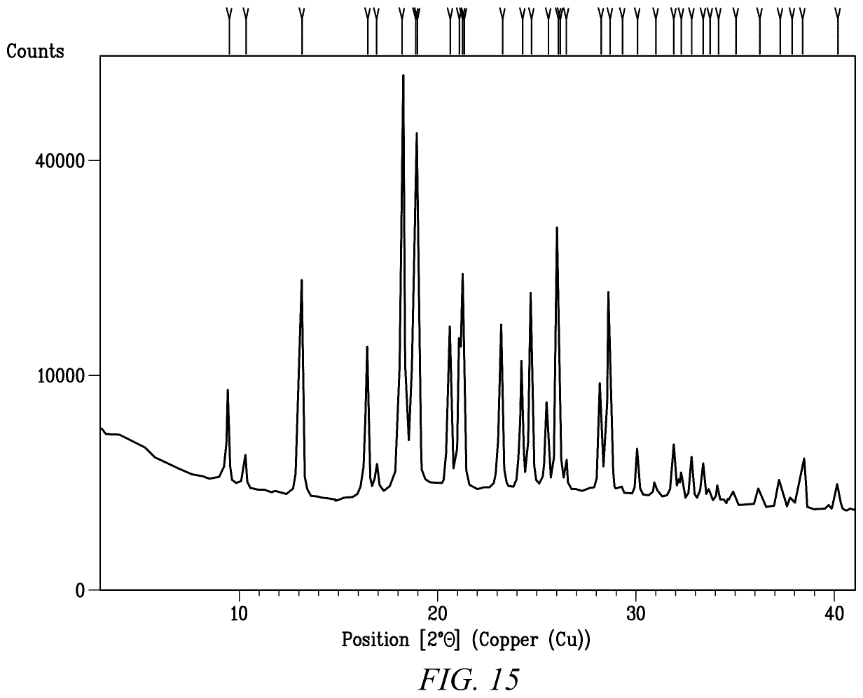

XRPD patterns were recorded on a PANalytical X Pert Pro diffractometer equipped with an X celerator detector using Ni filtered Cu Kα radiation at 45 kV and 40 mA. A 10 mm beam mask, and divergence (¼°) and anti-scatter (⅛°), and Soller (0.04 rad.) slits were inserted on the incident beam side. Receiving (5 mm) and Soller (0.04 rad.) slits and a Ni filter were inserted on the diffracted beam side. The X-ray powder pattern scan was collected from ca. 2 to 40° 20 with a 0.0080° step size and 96.06 sec counting time which resulted in a scan rate of approximately 0.5°/min. The sample was spread on a glass plate or a silicon zero background (ZBG) plate for the measurement. The sample was rotated at 15 revolutions/min on a PANalytical PW3065/12 Spinner. Measurement of the Si reference standard before the data collection resulted in values for 20 and intensity that were well within the tolerances of 28.38<20<28.50 and significantly greater than the minimum peak height of 1000 cps. The XRPD pattern of detomidine free base is shown in FIG. 15.

### Example 5c: DSC—Differential Scanning Calorimetry

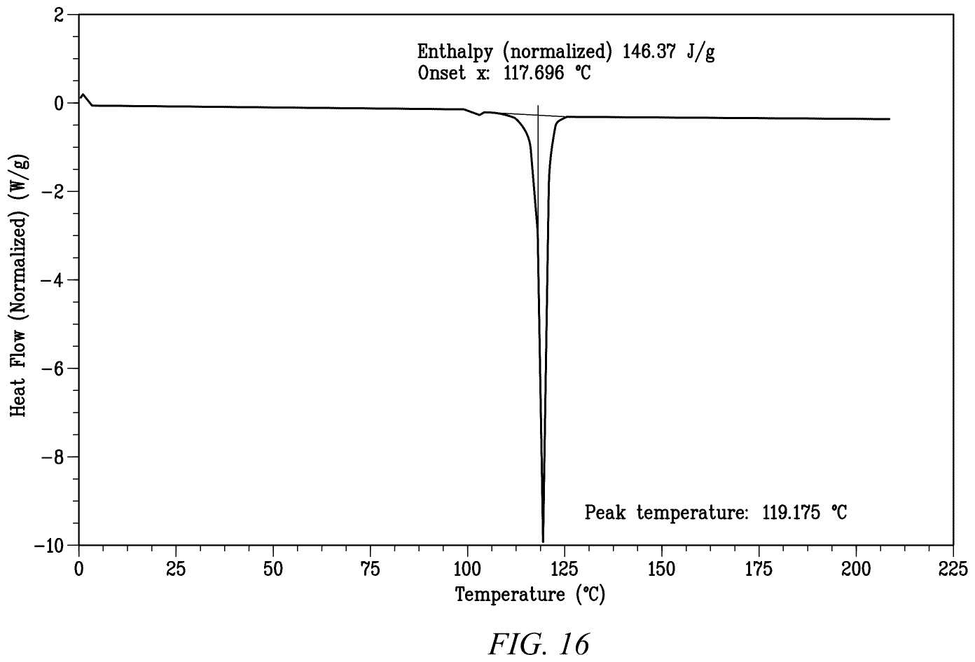

Thermal curves were acquired using a TA Discovery DSC unit. Solid samples of 5-20 mg were weighed into Tzero™ aluminum pinhole hermetically sealed pin hole pans. The DSC cell was then purged with nitrogen and the temperature heated at 10° C./min. from 0° to 200° C. Indium (Tm=156.6° C.; ΔHFus=28.45 J g-1) was used for calibration. The DSC thermogram of detomidine free base is shown in FIG. 16.

### Example 5d: TGA—Thermogravimetric Analyzer

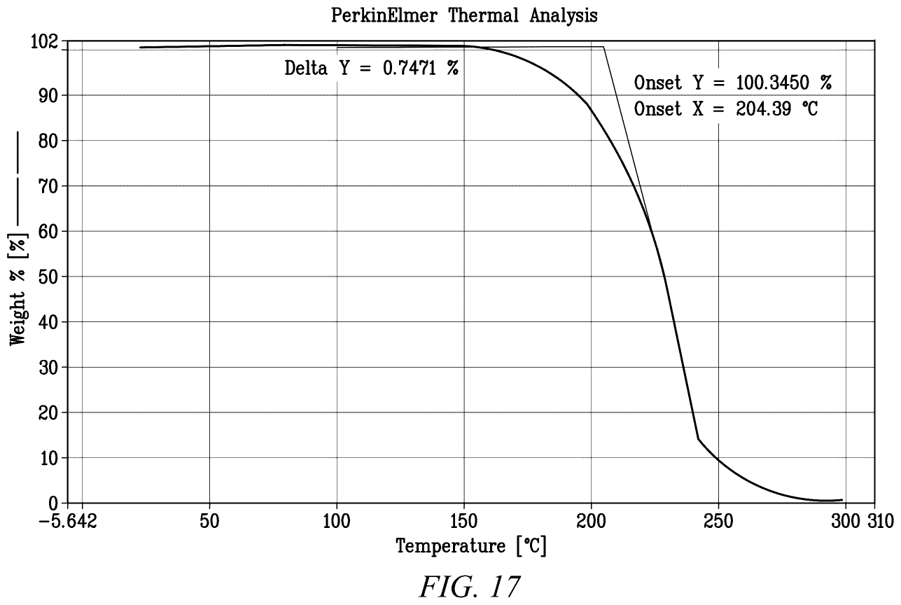

Thermal curves were acquired using a Perkin-Elmer Pyris 1 TGA unit running Pyris software version 6.0 calibrated with alumel (95% nickel, 2% manganese, 2% aluminum and 1% silicon), nickel and calcium oxalate monohydrate. TGA samples between 1-10 mg were monitored for percent weight loss as heated from 25 to 250° C. at 10° C./min in a furnace purged with Helium at ca. 50 mL/min. The percent weight loss to 150° C. is the value reported in Table 11 and the TGA thermogram of detomidine free base in FIG. 17.

Table 11 shows the first “25” peaks identified in an automatic peak search

| | | FWHM | | |
|---|---|---|---|---|
| Pos. [°2θ] | Height [cts] | Left [°2θ] | d-spacing [Å] | Rel. Int. [%] |
| 9.4369 | 6143.66 | 0.0708 | 9.37201 | 10.89 |
| 10.2822 | 1613.51 | 0.0787 | 8.60336 | 2.86 |
| 13.1267 | 19079.67 | 0.1023 | 6.74475 | 33.81 |
| 16.4182 | 11003.25 | 0.1023 | 5.39925 | 19.50 |
| 16.8720 | 1313.14 | 0.0945 | 5.25502 | 2.33 |
| 18.1807 | 56437.43 | 0.1496 | 4.87959 | 100.00 |
| 18.8002 | 38924.53 | 0.0866 | 4.72018 | 68.97 |
| 18.9037 | 43045.45 | 0.0866 | 4.69456 | 76.27 |
| 20.5659 | 12491.60 | 0.0787 | 4.31875 | 22.13 |
| 21.0058 | 9893.49 | 0.0866 | 4.22929 | 17.53 |
| 21.2170 | 19555.98 | 0.0864 | 4.18419 | 34.65 |
| 21.2634 | 12727.98 | 0.0384 | 4.18555 | 22.55 |
| 23.1638 | 12929.43 | 0.1728 | 3.83676 | 22.91 |
| 24.1591 | 9445.17 | 0.0960 | 3.68089 | 16.74 |
| 24.2191 | 6714.47 | 0.0384 | 3.68103 | 11.90 |
| 24.6326 | 16859.78 | 0.1632 | 3.61120 | 29.87 |
| 25.4240 | 5466.94 | 0.1056 | 3.50055 | 9.69 |
| 25.9866 | 26594.20 | 0.1056 | 3.42603 | 47.12 |
| 26.0536 | 17505.31 | 0.0384 | 3.42587 | 31.02 |
| 26.4132 | 1532.75 | 0.0576 | 3.37165 | 2.72 |
| 28.1107 | 7071.67 | 0.1248 | 3.17180 | 12.53 |
| 28.5554 | 17443.62 | 0.1056 | 3.12340 | 30.91 |
| 29.1749 | 265.43 | 0.1536 | 3.05848 | 0.47 |
| 30.0116 | 2373.54 | 0.0960 | 2.97508 | 4.21 |
| 30.8465 | 598.97 | 0.1152 | 2.89644 | 1.06 |

### Example 6: Topical Formulations Containing Detomidine HCl

Topical formulations containing detomidine HCl were prepared. The specific form of Detomidine HCl used in each preparation are also listed.

TABLE 11
Detomidine HCl formulations

| | | | | | | |
|---|---|---|---|---|---|---|
| Formulation | A | B | C | D | E | F |

Detomidine HCl

| | | | | | | |
|---|---|---|---|---|---|---|
| | 0.10% | 0.33% | 1.00% | 0.10% | 0.33% | 1.00% |
| Table 6, Batch # | i | i | i | iv | iv | iv |

excipients

| | | | | | | |
|---|---|---|---|---|---|---|
| Hydroxyethyl | 1.75% | 1.50% | 1.50% | 1.75% | 1.75% | 1.75% |
| cellulose | | | | | | |
| Glycerin | 12% | 20% | 20% | 0.30% | 0.33% | 3% |
| Propylene | 3% | — | — | 0.10% | 1% | 1% |
| Glycol | | | | | | |
| PEG 3350 | 7% | 12% | 12% | — | — | — |
| Benzalkonium | 0.02% | 0.02% | 0.02% | 0.02% | 0.02% | 0.02% |
| Chloride | | | | | | |

buffer

| | | | | | | |
|---|---|---|---|---|---|---|
| NaOH | — | — | 0.13% | — | — | — |
| Phosphate buffer | — | — | 10% | — | — | — |
| 0.1M pH 6.4 | | | | | | |

| | | | | |
|---|---|---|---|---|
| Buffer | Tris | Water | citrate buffer | citrate |
| Ad 100% | 0.4M | | 0.05M | buffer |
| | pH 8.2 | | pH 5.5 | 0.2M |
| | | | | pH |
| | | | | 5.2 |

### Example 7: Manufacturing Scale Topical Formulations Containing Detomidine HCl

15-36 kg batches of detomidine HCl gel were manufactured of formulations A-F as described in Example 6. Scaled up formulation A used material from Table 6, Batch #v, whereas scaled up formulations B-F used material from Table 6, Batch #iv.

For each scaled up product, each of the excipients, apart from hydroxyethyl cellulose, were dissolved in the formulation's buffer to create a co-solvent solution. Detomidine HCl was then dissolved in excipient solvents and added to this solution and mixed. Once it was confirmed that detomidine HCl had been fully dissolved, hydroxyethyl cellulose was introduced and the formulation mixed until a uniform gel formed.

### Example 8: Foreign Particle Identification in Manufacturing Scale Topical Formulations

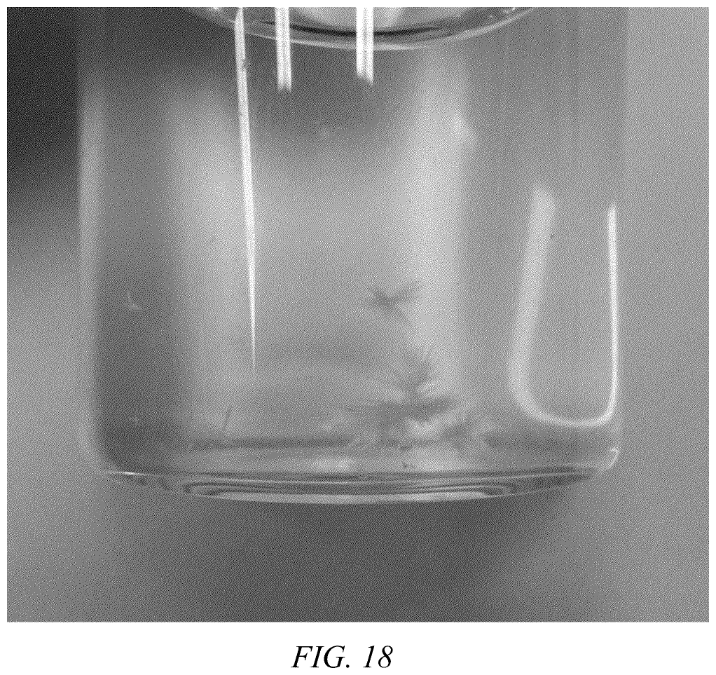

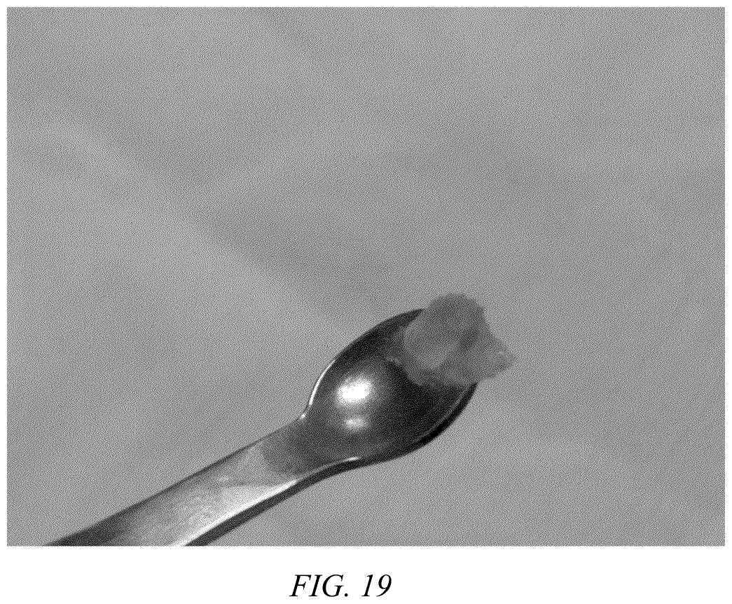

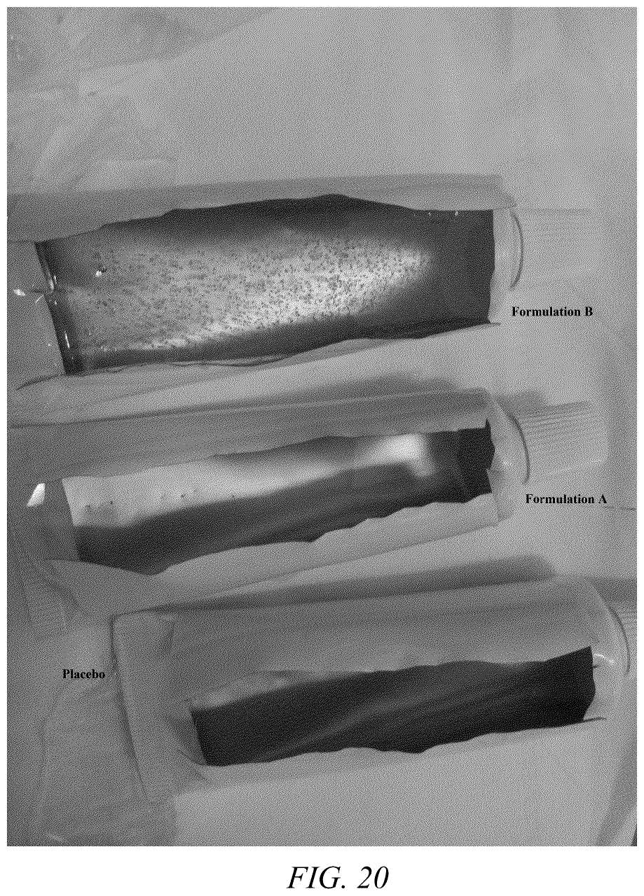

Upon completion of manufacture, the scaled up product of formulations A-C, as described in Example 7 were examined visually for their gel consistency. The scaled up products of formulations B and C were observed to have large quantities of foreign particles mixed together with the gel. Scaled up formulation B, together with the foreign particles is shown in FIG. 18 and an example of the isolated foreign particle is shown in FIG. 19.

An assay analysis of the formulations revealed that the foreign particles accounted for 4% of the total detomidine content of the manufactured product. Raman spectrography, comparing to the compound of Example 5, identified the foreign particles to be crystalline detomidine base.

In contrast, in scaled up formulation A, no particles were identified and the detomidine content as assayed by HPLC was found to be 100%. FIG. 20 shows scaled up formulations A and B where the manufactured gel has been filled into sample tubes, which have then been cut open along their length to allow for visual inspection of their content.

### Example 9: Aldehyde Impurity Assessment

Stock solutions of 7.4 wt % detomidine HCl in water were prepared using material from Table 6, Batch #iv and v and the samples were then kept exposed to air at room temperature for 2 years. HPLC analysis of RRT=0.38 content at time zero and after 2 years are provided in Table 12.

TABLE 12
RRT = 0.38 stock solution content

| Detomidine | Initial RRT = 0.38 | RRT = 0.38 content |
|---|---|---|
| Batch # | content | at 2 years |
| iv | 0.2% | 1% |
| v | not detectable | 0.02% |

A further sample of material from Table 6, Batch #v was reacted with potassium permanganate in aqueous solution. RRT=0.38 was found in the reaction product at 0.14% at 3 hours after solution preparation.

Together, these results suggest that the aldehyde impurity, 3-((1H-Imidazol-4-yl)methyl)-2-methylbenzaldehyde, might be generated in an aqueous environment by oxidation and might thereby affect an water based pharmaceutical composition's long term stability.

### Example 10: Stability of Topical Formulations Containing Detomidine HCl

Topical detomidine HCl formulations were manufactured of formulations D-F as described in Example 6 using material from Example 4c in place of Table 6, Batch #iv. Preliminary up-to 3 month stability studies with the formulations filled and sealed into glass vials were performed for initial assessment of their stability. The batches were tested at long-term conditions (25° C.), accelerated conditions (40° C.) and additionally at extreme conditions (55° C.) for faster assessment of stability of the prototypes. The stability results for these batches are provided in Table 13.

TABLE 13
Stability results of RRT = 0.38

| For- | | 25° C. | 40° C. | | | 55° C. | | | |
|---|---|---|---|---|---|---|---|---|---|
| mulation | T₀ | 3M | 1M | 2M | 3M | 13d | 1M | 2M | 3M |
| D | 0.02 | 0.02 | 0.02 | 0.02 | 0.02 | 0.02 | 0.06 | 0.03 | 0.12 |
| E | 0.01 | 0.01 | 0.01 | 0.01 | 0.01 | 0.01 | 0.01 | 0.01 | 0.01 |
| F | 0.00 | 0.00 | 0.03 | 0.00 | 0.00 | 0.01 | — | 0.01 | 0.01 |

### Example 11: Stability of Topical Formulations Containing Detomidine HCl

Topical detomidine HCl formulations D-F of Example 7 were filled and sealed in laminated tubes and assessed in stability studies. The batches were tested at long-term conditions (25° C./60% RH) and accelerated conditions (40° C./75% RH). The stability results for these batches are provided in Table 14. Further validation analyses of these studies identified that the Relative Response Factor (RRF) of the aldehyde impurity, 3-((1H-Imidazol-4-yl)methyl)-2-methylbenzaldehyde, was 1.0.

TABLE 14
Stability results of RRT = 0.38

| | | 25° C. | | | | | | 40° C. | | | |
|---|---|---|---|---|---|---|---|---|---|---|---|
| Formulation | T₀ | 1M | 2M | 3M | 6M | 9M | 12M | 1M | 2M | 3M | 6M |
| D | 0.18 | 0.19 | 0.19 | 0.19 | 0.18 | 0.20 | 0.16 | 0.18 | 0.19 | 0.18 | 0.18 |
| E | 0.18 | 0.18 | 0.18 | 0.18 | 0.18 | 0.19 | 0.17 | 0.18 | 0.19 | 0.18 | 0.18 |
| F | 0.18 | 0.18 | 0.18 | 0.18 | 0.18 | 0.19 | 0.16 | 0.18 | 0.18 | 0.18 | 0.17 |

What is claimed:

1. A method for providing prolonged, non-systemic treatment of a condition in a human subject in need thereof, comprising
topically administering to the skin of the human subject a formulation comprising
detomidine or a salt thereof in an amount of about 0.001 to about 3 wt %, based on the weight of the formulation, and
a carrier that is suitable for topical administration to human skin,
wherein the carrier optionally comprises a water-miscible solubilizer that is present in an amount of 0.1 to 40 wt %, based on the weight of the formulation,
wherein the formulation has a pH of 4.5 to 9, and,
wherein the formulation comprises less than about 1 wt % of 3-((1H-Imidazol-4-yl)methyl)-2-methylbenzaldehyde at least 6 months after its manufacture.

2. The method of claim 1, wherein the condition is pain.

3. The method of claim 1, wherein the formulation comprises about 0.1 to about 1% detomidine or a salt thereof.

4. The method of claim 1, wherein the formulation comprises a water-miscible solubilizer.

5. The method of claim 4, wherein the water-miscible solubilizer comprises a diol or a polyol.

6. The method of claim 4, wherein the water-miscible solubilizer comprises glycerol, propylene glycol, or a combination thereof.

7. The method of claim 1, wherein the formulation further comprises a buffer that is effective to maintain the formulation at a pH of about 4.5 to about 7.

8. The method of claim 7, wherein the pH of the formulations is about 5 to about 6.

9. The method of claim 1, wherein the salt of detomidine is detomidine HCl.

10. The method of claim 1, wherein the formulation comprises less than about 1 wt % of 3-((1H-Imidazol-4-yl)methyl)-2-methylbenzaldehyde at least 12 months after its manufacture.

11. The method of claim 1, wherein the formulation comprises less than about 0.2 wt % of 3-((1H-Imidazol-4-yl)methyl)-2-methylbenzaldehyde at least 12 months after its manufacture.

12. The method of claim 1, wherein the topical administration of the formulation to the skin of the human subject induces formation of a depot of detomidine or the salt thereof in the human subject's skin.

13. The method of claim 1, wherein topical administration of the formulation to the skin of the human subject once- or twice-daily for up to four days results in a blood plasma concentration in the human subject that is less than that required to achieve a systemic therapeutic effect of the detomidine or salt thereof.

14. A method for providing prolonged, non-systemic treatment of a condition in a human subject in need thereof, comprising
topically administering to the skin of the human subject a pharmaceutical formulation comprising
detomidine or a salt thereof in an amount of about 0.001 to about 3 wt %, based on the weight of the formulation, and
a carrier that is suitable for topical administration to human skin,
wherein the carrier optionally comprises a water-miscible solubilizer that is present in an amount of 0.1 to 40 wt %, based on the weight of the formulation,
wherein the formulation has a pH of 4.5 to 9, and,
wherein the formulation is substantially free of 3-((1H-Imidazol-4-yl)methyl)-2-methylbenzaldehyde.

15. The method of claim 14, wherein the condition is pain.

16. The method of claim 14, wherein the composition comprises about 0.1 to about 1 wt. % of the detomidine or the salt thereof.

17. The method of claim 14, wherein the formulation comprises a water-miscible solubilizer.

18. A method for providing prolonged, non-systemic treatment of a condition in a human subject in need thereof, comprising
topically administering to the skin of the human subject a pharmaceutical formulation comprising
detomidine or a salt thereof in an amount of about 0.001 to about 3 wt %, based on the weight of the formulation, and
a carrier that is suitable for topical administration to human skin,
wherein the carrier optionally comprises a water-miscible solubilizer that is present in an amount of 0.1 to 40 wt %, based on the weight of the formulation,
wherein the formulation has a pH of 4.5 to 9, and,
wherein the topical formulation comprises less than 4% of the total detomidine content as crystalline detomidine base.

19. The method according to claim 18, wherein the condition is pain.

20. The method according to claim 18, wherein the condition is pruritus.
